SEQUENCE LISTING (4 sequences)

SEQ ID NO: 1 - RNA, 1653 nt, synthetic construct
  misc_feature 1..1653: note = Firefly luciferase (Luc) mRNA
  source 1..1653: mol_type = other RNA
atggaggacgccaagaacatcaagaagggccccgcccccttctaccccctggaggacggcacagccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgcctcatagaaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccctctgagcaaggaggtgggagaggccgtggccaagcggttccacctgcccggcatcagacagggctacggcctgaccgagaccaccagcgccatcctgatcacccccgagggcgacgacaagcctggcgccgtgggcaaggtggtgcccttcttcgaggccaaggtggtggacctggacaccggcaagaccctgggcgtgaaccagaggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcctgatgacgatgccggagagctgcctgccgccgtggtggtgctggagcacggcaagaccatgaccgagaaggagatcgtggactacgtggccagccaggtgaccaccgccaagaagctgaggggcggcgtggtgttcgtggacgaggtgcccaagggcctgaccggcaagctggacgcccggaagatccgggagatcctgatcaaggccaagaagggcggcaagatcgccgtgtga

SEQ ID NO: 2 - RNA, 1161 nt, synthetic construct
  misc_feature 1..1161: note = Ovalbumin (OVA) mRNA
  source 1..1161: mol_type = other RNA
atgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccggacccagatcaacaaggtggtgcggttcgacaagctgcccggcttcggcgacagcatcgaggcccagtgcggcaccagcgtgaacgtgcacagcagcctgcgggacatcctgaaccagatcaccaagcccaacgacgtgtacagcttcagcctggccagccggctgtacgccgaggagcggtaccccatcctgcccgagtacctgcagtgcgtgaaggagctgtaccggggcggcctggagcccatcaacttccagaccgccgccgaccaggcccgggagctgatcaacagctgggtggagagccagaccaacggcatcatccggaacgtgctgcagcccagcagcgtggacagccagaccgccatggtgctggtgaacgccatcgtgttcaagggcctgtgggagaagaccttcaaggacgaggacacccaggccatgcccttccgggtgaccgagcaggagagcaagcccgtgcagatgatgtaccagatcggcctgttccgggtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcggaagatcaaggtgtacctgccccggatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgggaggtggtgggcagcgccgaggccggcgtggacgccgccagcgtgagcgaggagttccgggccgaccaccccttcctgttctgcatcaagcacatcgccaccaacgccgtgctgttcttcggccggtgcgtgagcccctga

SEQ ID NO: 3 - RNA, 1644 nt, Mus musculus
  source 1..1644: mol_type = mRNA
atgccatctcctctccctgtctccttcctcctctttcttaccttagtaggaggcaggccccagaagtccttactggtggaggtagaagagggaggcaatgttgtgctgccatgcctcccggactcctcacctgtctcttctgagaagctggcttggtatcgaggtaaccagtcaacacccttcctggagctgagccccgggtcccctggcctgggattgcacgtggggtccctgggcatcttgctagtgattgtcaatgtctcagaccatatggggggcttctacctgtgccagaagaggccccctttcaaggacatctggcagcctgcctggacagtgaacgtggaggatagtggggagatgttccggtggaatgcttcagacgtcagggacctggactgtgacctaaggaacaggtcctctgggagccacaggtccacttctggttcccagctgtatgtgtgggctaaagaccatcctaaggtctggggaacaaagcctgtatgtgcccctcgggggagcagtttgaatcagagtctaatcaaccaagatctcactgtggcacccggctccacactttggctgtcctgtggggtaccccctgtcccagtggccaaaggctccatctcctggacccatgtgcatcctaggagacctaatgtttcactactgagcctaagccttgggggagagcacccggtcagagagatgtgggtttgggggtctcttctgcttctgccccaagccacagctttagatgaaggcacctattattgtctccgaggaaacctgaccatcgagaggcacgtgaaggtcattgcaaggtcagcagtgtggctctggctgttgagaactggtggatggatagtcccagtggtgactttagtatatgtcatcttctgtatggtttctctggtggcttttctctattgtcaaagagcctttatcctgagaaggaaaaggaagcgaatgactgaccccgccaggagattcttcaaagtgacgcctccctcgggaaacgggacccagaaccagtacgggaatgtgctctcccttcctacatctacctctggccaggcccatgctcagcgttgggctgctggcctagggagtgtccctgggtcctatggaaatccacgcattcaagtccaggatactggagctcagagccatgaaacaggactggaagaagaaggggaggcctatgaagagccagacagcgaggagggctctgaattctatgagaacgactccaaccttgggcaggaccaggtttcccaggatgggagtggctatgagaaccccgaggatgagcccatgggtccagaggaagaagactccttctccaatgctgagtcttatgaaaatgcagatgaggagctggcccaaccagttggcaggatgatggacttcctgagcccccatgggtctgcgtgggaccccagccgggaagcatcctcgcttgggtcccagtcctatgaagatatgagagggatcctctatgcagctcctcagctccactcaattcagtccggtcccagtcatgaagaagatgcagactcttatgaaaacatggataagtctgacgacctagaaccagcatgggaaggagagggccacatggggacttggggaaccacgtga

SEQ ID NO: 4 - protein, 8 aa, synthetic construct
  REGION 1..8: note = SIINFEKL OVA peptide
SIINFEKL

(19) United States
(12) Patent Application Publication
(10) Pub. No.: US 2025/0235532 A1
(43) Pub. Date: Jul. 24, 2025

(54) POLYMER-LIPID HYBRID NANOPARTICLES COMPRISING A LIPID AND A BLOCK COPOLYMER AS WELL AS METHODS OF MAKING AND USES THEREOF

(71) Applicant: ACM BIOLABS PTE LTD, Singapore (SG)

(72) Inventors: Madhavan NALLANI, Singapore (SG); Teck Wan CHIA, Singapore (SG); Shaoqiong LIU, Singapore (SG); Gaurav SINSINBAR, Singapore (SG); Jian Hang LAM, Singapore (SG)

(73) Assignee: ACM BIOLABS PTE LTD, Singapore (SG)

(21) Appl. No.: 18/840,856

(22) PCT Filed: Feb. 23, 2023
(86) PCT No.: PCT/EP2023/054613

§ 371 Date: Aug. 22, 2024

(30) Foreign Application Priority Data
Feb. 23, 2022 (EP) 22158324.8

(51) Int. Cl. A61K 39/385; A61K 9/51; A61K 31/7105; A61K 38/46; A61K 39/00; A61K 39/12; A61K 39/135; A61K 39/145; A61K 39/215; A61K 39/245; A61K 48/00; A61P 37/04

(52) U.S. Cl. CPC A61K 39/385; A61K 9/5153; A61K 31/7105; A61K 38/465; A61K 39/0008; A61K 39/0011; A61K 39/12; A61K 39/135; A61K 39/145; A61K 39/215; A61K 39/245; A61K 48/0041; A61P 37/04; A61K 2039/54; A61K 2039/541

(57) ABSTRACT

The present invention relates to a polymer-lipid hybrid nanoparticle comprising a lipid and a block copolymer, wherein the amount of said lipid, expressed in mole percentage (mole %) present in the polymer-lipid hybrid nanoparticle, wherein the mole percentage refers to the total amount of all components that form the polymer-lipid nanoparticle, is greater than the amount of said block copolymer, expressed in mole percentage, present in the polymer-lipid hybrid nanoparticle. The invention also relates to such a polymer-lipid hybrid nanoparticle further comprising a soluble encapsulated antigen, wherein said soluble encapsulated antigen is a protein and/or polynucleotide. The invention further relates to a method of encapsulating such an antigen in such a polymer-lipid hybrid nanoparticle as well as to a composition comprising such a polymer-lipid hybrid nanoparticle and uses of such a polymer-lipid hybrid nanoparticle and/or composition as a vaccine, a pharmaceutical, means of targeting cells, tissues and/or organs and/or non-viral delivery system capable of delivering nucleotides to inside a cell.

### CROSS-REFERENCE TO RELATED APPLICATIONS

The present application claims the right of priority of European patent application 22158324 filed with the European Patent Office on 23 Feb. 2022, the entire content of which is incorporated herein for all purposes.

### SEQUENCE LISTING

This application contains a Sequence Listing in computer readable form, which is incorporated herein by reference.

### TECHNICAL FIELD

The present invention relates to a polymer-lipid hybrid nanoparticles comprising a lipid and a block copolymer, wherein the amount of said lipid expressed in mole percentage (mole %) present in the polymer-lipid hybrid nanoparticle is greater than the amount of said block copolymer expressed in mole percentage present in the polymer-lipid hybrid nanoparticle. The invention also relates to such a polymer-lipid hybrid nanoparticle/s further comprising a soluble encapsulated antigen/s, wherein said soluble encapsulated antigen/s is a protein/s and/or polynucleotide/s. The invention further relates to a method of encapsulating such an antigen/s in such a polymer-lipid hybrid nanoparticles as well as to a composition/s comprising such a polymer-lipid hybrid nanoparticle/s and uses of such a polymer-lipid hybrid nanoparticle/s and/or composition/s as a vaccine, a pharmaceutical, means of targeting cells, tissues and/or organs and/or non-viral delivery system capable of delivering nucleotides, e.g., to inside a cell. In particular, in the course of the present invention, synthetic polymer-lipid hybrid nanoparticle comprising block copolymer PBD-PEO (non-degradable) or Poly(ε-caprolactone)-poly (ethylene glycol) (PCL-PEO) (biodegradable) have been explored as novel platform for polynucleotide (e.g., mRNA) delivery.

### BACKGROUND OF THE INVENTION

Although immunization is a well-established process, there are differences in the response level elicited between different immunogens or antigens. For example, membrane proteins form a class of antigens that produce a low response level, which in turn means that large amounts of membrane proteins are required to generate or elicit an immune response to the desired level. Membrane proteins are notoriously difficult to synthesize and are insoluble in water without the presence of a detergent. This makes it expensive and difficult to obtain membrane proteins in sufficient quantity for immunization. Furthermore, membrane proteins require proper folding to function correctly. The immunogenicity of correctly folded native membrane proteins is typically much better than that of their solubilized forms, which may not be folded in a physiologically relevant manner. Thus, even though adjuvants may be used to boost the immunogenicity of such solubilized antigens, it is an inefficient method that does not provide too much of an advantage (e.g., WO2014/077781A1).

Although transfected cells and lipid-based systems have been used to present membrane protein antigens to increase the chances of isolating antibodies that may be efficient in vivo, these systems are often unstable (e.g., oxidation sensitive), tedious and costly. Moreover, the current state of the art for such membrane protein antigens is to use inactive virus-like particles for immunization.

On the other hand, vaccines are the most efficient way to prevent diseases, mainly infectious diseases [e.g., Liu et al., 2016]. As of today, most of the licensed vaccines are made of either live or killed viruses. Despite their effectiveness in generating a humoral response (an antibody mediated response) to prevent viral propagation and entry into cells, safety of such vaccines remains a concern. In the past few decades, scientific advances have helped to overcome such issues by engineering vaccine vectors that are non-replicating recombinant viruses. In parallel, protein based antigens or sub-unit antigens have been explored as safer alternatives. However, such protein based vaccines typically illicit poor immune (both humoral and cellular response). To improve immunogenic properties of antigens, several approaches have been used. For example, microencapsulation of antigens into polymers has been investigated extensively, although it did enhance the immunogenicity, aggregation and denaturing of antigens remain unsolved [e.g., Hilbert et al., 1999]. Furthermore, adjuvants (e.g., oil in water emulsions or polymer emulsions) [e.g., U.S. Pat. No. 9,636,397B2, US2015/0044242 A1] are used together with antigens to elicit a more pronounced humoral and cellular response. Despite these advances, they are less efficient in uptake and cross-presentation. To promote cross-presentation, based on the available information of the immune system during infection by viruses, viral like particles that mimic such properties have been exploited. Synthetic architectures such as liposomes with encapsulated antigens are particularly attractive. Liposomes are unilamellar self-assembling structures made of lipids and, cationic liposomes are more attractive and promising as delivery vehicles because of their efficient uptake by Antigen Presenting Cells (APCs) [e.g., Maji et al., 2016]. Furthermore, it allows integrating immunomodulators such as Monophosphoryl Lipid A (MPL), CpG oligodeoxynucleotide, that are toll-like receptor (TLR) agonists which stimulate immune cells through receptors. Despite these opportunities of such delivery vehicles, one of the limiting factors is stability of liposomes in the presence of serum components. By PEGylations, loading with high melting temperature lipids, stability issues of liposomes are somewhat reduced with and one such well characterized example being inter bilayered-crosslinked multilamellar vesicles (ICMVs), formed by stabilizing multilamellar vesicles with short covalent crosslinks linking lipids [e.g., Moon et al., 2011]. Other nanoparticle architectures have led to successful immunisations using nanodiscs [e.g., Kuai et al., 2017] or pH sensitive particles [e.g., Luo et al., 2017]. But such strategies either still requires adjuvants or are not as efficient outside the prototypical Ovalbumin (OVA) models.

In addition, polymersomes, offer as a stable alternative for liposomes and they have been used to integrate membrane proteins to elicit immune response [e.g., Quer et al., 2011, WO2014/077781A1]. Protein antigens were also encapsulated in a chemically altered membrane of the polymersome (however oxidation-sensitive membranes) to release antigens and the adjuvants to dendritic cells [e.g., Stano et al., 2013].

On the other hand messenger RNA (mRNA) has arisen as a promising strategy for prevention and treatment of various diseases including infections, cancer and gene disorders. However, the clinical translation of mRNA therapeutic is impeded by its instability and inefficient in vivo delivery. Recently, advanced lipid nanoparticles (LNP) systems have demonstrated potency in preclinical trials and successfully entered the clinics. For instance, LNP-Onpattro (LNP-ON) containing DMG-PEG, DSPC, MC3 and Chol (at a ratio 1.5:10.0:50: 38.5) has been approved by the FDA in 2018, where a therapeutic siRNA (pastisiran) was encapsulated for treatment of hereditary mediated amyloidosis. To date, many other LNPs have been developed for mRNA delivery. Notably, mRNA-1273 and BNT162b have been used in clinics globally for the prevention of coronavirus disease 2019 (COVID-19). Despite some positive results, it remains a challenge to maintain the long-term stability and potency of mRNA loaded LNP. For instance, mRNA-1273 and BNT162b are recommended to be stored at −80° C. and −20° C., respectively. As cold chain transportation and storage are not available in many areas, there is an urgent need to develop therapeutics with enhanced long-term stability.

Accordingly, despite this progress made by the use of polymers, there remains a need to provide for efficient and stable uptake, delivery and/or stable cross-presentation delivery vehicles/systems and methods based thereon that overcome, or at least alleviate, the above problems as well as possess an improved functionality inter alia in that they are also capable of eliciting a CD8⁽⁺⁾ T cell-mediated immune response, which is particularly important in treatment and/or prevention of infectious diseases, cancers and autoimmune diseases.

### SUMMARY OF THE INVENTION

The present invention relates to a polymer-lipid hybrid nanoparticle comprising a lipid and a block copolymer, wherein the amount of said lipid, expressed in mole percentage (i.e., a mole %) present in the polymer-lipid hybrid nanoparticle, wherein the mole percentage refers to the total amount of all components that form the polymer-lipid nanoparticle, is greater than the amount of said block copolymer, expressed in mole percentage, present in the polymer-lipid hybrid nanoparticle.

The present invention further relates to such a polymer-lipid hybrid nanoparticle, wherein the lipid (e.g., ionizable lipid) is selected from a group consisting of: an ionizable lipid DLin-MC3-DMA (also referred to as MC3) and an ionizable lipid C12-200. The present invention further relates to such a polymer-lipid hybrid nanoparticle, wherein the block copolymer is selected from a group consisting of: poly(butadiene)-b-poly(ethylene glycol) (PBD-PEO) block copolymer, poly caprolactone (PCL)-PEO block copolymer, poly(Lactide-co-glycolide) (PLGA)-PEO (e.g., with various LA to GA ratios) and DMG-PEG block copolymer. The present invention further relates to such a polymer-lipid hybrid nanoparticle, wherein a mole % ratio of the lipid to the block copolymer is between 31.8 to 12 and about 35 to 2.5. The present invention further relates to such a polymer-lipid hybrid nanoparticle, further comprising a stabilizer, e.g., comprising or consisting of cholesterol (also referred to as CHOL). The present invention further relates to such a polymer-lipid hybrid nanoparticle, further comprising another lipid, wherein said another lipid is selected from a group consisting of: DMPC, DSPC, DOPE, DOTAP, DODAP, DOTMA, DODMA, DDA, 18:1 PA (1,2-dioleoyl-sn-glycero-3-phosphate), 14:0 PA (1,2-dimyristoyl-sn-glycero-3-phosphate), 18:1 BMP (bis(monooleoylglycero)phosphate). The present invention further relates to such a polymer-lipid hybrid nanoparticle, consisting of: (i) PBD-PEO, MC3, CHOL; (ii) PBD-PEO, C12-200, CHOL; (iii) PBD-PEO, DOPE, C12-200, CHOL; (iv) PBD-PEO, DOPE, C12-200, CHOL; (v) PBD-PEO, DOPE, C12-200, CHOL; (vi) PBD-PEO, DOPE, C12-200, CHOL; (vii) DMG-PEG, DSPC, MC3, CHOL; (viii) PCL-PEO, DMPC, MC3, CHOL; (ix) PCL-PEO, DMPC, MC3, CHOL; (x) PCL-PEO, DMPC, MC3, CHOL; or (xi) PCL-PEO, DMPC, MC3, CHOL. The present invention further relates to such a polymer-lipid hybrid nanoparticle, further comprising a soluble encapsulated antigen, wherein said soluble encapsulated antigen is a protein and/or polynucleotide.

The present invention further relates to a composition comprising such a polymer-lipid hybrid nanoparticle.

The present invention further relates to a method of delivering nucleotide/s to inside a cell without using viral vector/s as delivery means, said method comprising: (i) providing the polymer-lipid hybrid nanoparticle and/or composition of the present invention; and (ii) contacting said polymer-lipid hybrid nanoparticle and/or composition with a cell.

Illustrative polymer-lipid hybrid nanoparticles of the present invention exhibit favorable physicochemical properties and/or superior encapsulation efficiency (˜100%). In comparison to benchmark LNP-ON (i.e., LNP-Onpattro or LNP-ONP, which can be used interchangeably herein), the polymer-lipid hybrid nanoparticles of the present invention outperform and enhance in vitro transfection efficacy and/or long term thermostability of polynucleotides (e.g., mRNA), Moreover, polymer-lipid hybrid nanoparticles formulations of the present invention display less cytotoxicity as compared to benchmark LNP-ON. Furthermore, illustrative polymer-lipid hybrid nanoparticles formulations of the present invention can strongly activate cDC1 and cDC2 in the lymph nodes to promote antigen surface presentation. Taken together, the present invention provides a novel class of polymer lipid hybrid nanoparticles with efficient protein and antigen expression as well as enhanced thermostability, which makes them suitable for delivery of therapeutic mRNA over a wide range of diseases.

Therefore, the present invention satisfies this demand by provision of stable polymer-lipid hybrid nanoparticles comprising a lipid and a block copolymer as described herein, methods based thereon as well as methods for their production and compositions comprising such a polymer-lipid hybrid nanoparticle, described herein, characterized in the claims and illustrated by the appended Examples and Figures.

### Overview of the Sequence Listing

SEQ ID NO: 1 is an exemplary firefly luciferase (Luc) mRNA sequence derived from Photinus pyralis.
SEQ ID NO: 2 is an exemplary Ovalbumin (OVA) mRNA (https://www.trilinkbiotech.com/media/folio3/productattachments/product_insert/ova_orf_catno_I -7210_I-7610_. txt).
SEQ ID NO: 3 is an exemplary Mus musculus CD19 mRNA sequence.
SEQ ID NO: 4 is an exemplary OVA peptide.

### BRIEF DESCRIPTION OF THE DRAWINGS

FIG. 1 shows cryo-TEM images (A, B) and particle size (C) of exemplary polymer-lipid hybrid nanoparticles (BNPs) of the present invention prepared by solvent dispersion method from the ionizable lipid DLin-MC3-DMA and PBD-PEO block copolymer and encapsulating Luciferase mRNA.

FIG. 2 shows cryo-TEM images (A, B, C) and particle size (D) of exemplary polymer-lipid hybrid nanoparticles (BNPs) of the present invention prepared by solvent dispersion method from the ionizable lipid DLin-MC3-DMA and PBD-PEO block copolymer and encapsulating Ovalbumin mRNA.

FIG. 3 shows Cryo-TEM images of exemplary polymer-lipid hybrid nanoparticles. FIG. 3A shows a Cryo-TEM image of exemplary BNP-002 polymer-lipid hybrid nanoparticles of the present invention prepared by the mixing method (alternative methods can for example be a T-Mixer method, homogenization and/or microfluidic chip-based mixing method) from the ionizable lipid DLin-MC3-DMA and PBD-PEO block copolymer and encapsulating Luciferase mRNA. FIG. 3B shows a Cryo-TEM image of exemplary BNP-008 polymer-lipid hybrid nanoparticles of the present invention prepared by the mixing method from the ionizable lipid C12-200 and PBD-PEO block copolymer and encapsulating Luciferase mRNA. FIG. 3C shows a Cryo-TEM image of exemplary PCL-008 polymer-lipid hybrid nanoparticles of the present invention prepared by the mixing method from the ionizable lipid DLin-MC3-DMA and PCL-PEO block copolymer and encapsulating Luciferase mRNA. FIG. 3D shows a Cryo-TEM image of exemplary PCL-012 polymer-lipid hybrid nanoparticles of the present invention prepared by the mixing method from the ionizable lipid DLin-MC3-DMA and PCL-PEO block copolymer and encapsulating Luciferase mRNA.

FIG. 4 shows electrophoretic analyses of exemplary polymer-lipid hybrid nanoparticles of the present invention. FIG. 4A shows an agarose gel image of Luciferase mRNA encapsulated by exemplary polymer-lipid hybrid nanoparticles BNP prepared by solvent dispersion method from the ionizable lipid DLin-MC3-DMA and PBD-PEO block copolymer compared to a control formulation. Luciferase mRNA remains intact after being encapsulated into BNP polymer-lipid hybrid nanoparticles. FIG. 4B shows an RNAse Protection Assay using gel electrophoresis analysis where exemplary polymer-lipid hybrid nanoparticles BNP samples were prepared by solvent dispersion method and stored at 4° C. for 2 weeks prior to the analysis.

FIG. 5 shows an in vitro transfection efficiency profiles in HEK293T cells after over 3 weeks (A, B) storage at 4° C. of Luciferase mRNA encapsulated by exemplary BNP polymer-lipid hybrid nanoparticles of the present invention prepared by solvent dispersion method from the ionizable lipid DLin-MC3-DMA and PBD-PEO block copolymer compared to a control formulation nanoparticles. Nanoparticles were prepared by solvent dispersion method, where N/P=27. N/P ratio: N (nitrogen) in the ionized cationic lipid and P (phosphorus) in mRNA.

FIG. 6 shows Ovalbumin protein expression in HEK293T cells post 24 h transfection (A, B) from OVA mRNA encapsulated by exemplary BNP polymer-lipid hybrid nanoparticles of the present invention prepared by solvent dispersion method from the ionizable lipid DLin-MC3-DMA and PBD-PEO block copolymer compared to a control formulation nanoparticles. Nanoparticles were prepared by solvent dispersion method, where N/P=27. N/P ratio: N (nitrogen) in the ionized cationic lipid and P (phosphorus) in mRNA.

FIG. 7 shows an agarose gel image of OVA mRNA encapsulated by exemplary BNP and PCL polymer-lipid hybrid nanoparticles of the present invention prepared by mixing method compared to a control formulation nanoparticles. All samples contain intact mRNA as no degradation observed from the gel.

FIG. 8 shows stability assay of Luciferase mRNA encapsulated by exemplary BNP and PCL polymer-lipid hybrid nanoparticles of the present invention after 1 month storage at 4° C.: In-vitro Luciferase mRNA nanoparticles (prepared by mixing method) transfection efficiency in HEK293T cells at 24 h post tranfection. * Using 25 ng Luc mRNA as reference. After 1 month at 4° C., LNP ON and BNP008 seems to have degraded while BNP002, PCL008, PCL012 unchanged.

FIG. 9 shows an in vitro cytotoxicity of Luc mRNA encapsulated by exemplary BNP and PCL polymer-lipid hybrid nanoparticles of the present invention (prepared by mixing method) against HEK293T cells after 24 h incubation.

FIG. 10 shows Ovalbumin protein expression (A, B) from OVA mRNA encapsulated by exemplary BNP and PCL polymer-lipid hybrid nanoparticles of the present invention (prepared by mixing method) in HEK293T cells post 24 h transfection.

FIG. 11 shows expression analysis of Luc mRNA. FIG. 11A shows an in vivo expression kinetics analysis of Luc mRNA encapsulated by exemplary BNP and PCL polymer-lipid hybrid nanoparticles of the present invention administrated to mice by intramuscular (IM) injections. FIG. 11B shows an ex vivo IVIS Bio-imaging of Luc mRNA delivered in ACM nanoparticles to mice administrated by IM. FIG. 11C shows an ex vivo IVIS Bio-imaging of Luc mRNA delivered in ACM nanoparticles to mice administrated by IV.

FIG. 12 shows tissue expression profiles of the Luc mRNA-encoded Protein in mice. FIG. 12A shows an ex vivo imaging analysis of tissue expression profiles of the Luc mRNA-encoded Protein in mice (at which time point—at 6 h) post IM injection. FIG. 12B shows tissue expression profiles of the Luc mRNA-encoded Protein in Mice at 6 h post IV Injection. FIG. 12C shows tissue expression profiles of the Luc mRNA-encoded Protein in Mice at 6 h post IV Injection as percentage of expression in individual tissues.

FIG. 13 shows activation of dendritic cells (DCs) in draining lymph nodes (Ovalbumin mRNA encapsulated by exemplary BNP polymer-lipid hybrid nanoparticles of the present invention).

FIG. 14 shows OVA peptide surface presentation (Ovalbumin mRNA encapsulated by exemplary BNP polymer-lipid hybrid nanoparticles of the present invention).

FIG. 15 shows Cas12a/gRNA encapsulation by exemplary BNP 002 polymer-lipid hybrid nanoparticles of the present invention. Cas12a and gRNA with ASF p52 were mixed at 250 nM concentration. The solution was incubated at RT for 10-15 mins for Cas12a to bind to gRNA. This was further encapsulated in the BNP-002 using the mixing method using PNI system with TFF 12 ml/min and FRR of 3:1 at 1 ml scale. After the formulations ware complete, DLS of the samples was done and rest of the samples was put on dialysis with PBS buffer. After dialysis sample was harvested and DLS was collected before and after sterile filtration.

FIG. 16 shows Dynamic light scattering (DLS) analysis of Cas12a/gRNA encapsulated by exemplary BNP polymer-lipid hybrid nanoparticles of the present invention prepared by mixing method.

FIG. 17 shows ACM-OVA mRNA vaccine adaptive immunity study. Mice immunised with ACM-OVA mRNA formulations. a. Immunisation and blood collection schedule. b, c. Circulating SIINFEKL-specific CD8+ T cells. d, e. Serum OVA IgG titre. Where appropriate, 2-or 1-way ANOVA with Tukey's multiple comparison was performed.

FIG. 18 shows Cryo-TEM images of exemplary BNP polymer-lipid hybrid nanoparticles of the present invention prepared by micro-fluidizer. (A) Cryo-TEM for BNP-002.2 (loaded with Luciferase mRNA), wherein BNP-002.2 show spherical nanoparticles (50˜150 nm) with amorphous structure. (B) Cryo-TEM for BNP-012 (loaded with Luciferase mRNA), wherein BNP-012 show predominant distribution: Multi-compartmental structure and wherein vesicles consist of heterogeneous structure (i.e., vesicles fusion; vesicles with buddy, vesicles buddy surrounding by bilayer). (C) Cryo-TEM for BNP-025 (loaded with Luciferase mRNA), wherein BNP-025 exhibit vesicle structure (30-150 nm) with relatively higher polydispersity.

FIG. 19 shows an agarose gel image of Luc mRNA loaded nanoparticles prepared by microfluidizer indicating that all samples contained intact mRNA as no degradation was observed in the gel.

FIG. 20 shows in vitro Luciferase mRNA nanoparticles transfection efficiency profiles in HEK293T cells indicating that all formulations had high expression of luciferase protein, which is comparable to that of LNP-ON and that BNP-002.2 demonstrated remarkably high in vitro transfection potency as compared to LNP-ON (p<0.05).

FIG. 21 shows a Luciferase Protein expression Biodistribution Percentage Profile via IV (intravenous) administration, wherein: BNP-002.2 yielded Luciferase protein accumulated in liver (54%), spleen (44.5%), 2.1% in the lung; BNP-012 led to Luciferase protein expression in liver (0.9%), while 1.4% in spleen, 92% in the lung; BNP-025 generated Luciferase protein in liver (2.7%), spleen (13%), 76% in the lung; BNP-012 and BNP-025 containing cationic lipids (DOTAP and DOTMA) generated luciferase protein predominately at lung.

FIG. 22 shows Tissue Expression Profiles (Raw value of Flux) of the Luc mRNA-encoded Protein in Mice 6 h Post Administration via IV (intravenous). Raw value of Flux (Photons s1); LNP ONP produce significantly higher Luc protein than other groups at liver; BNP-008 yielded significant higher Luc protein than other groups at spleen; The amount of Luc protein expression at lung among difference groups is similar.

FIG. 23 shows Luciferase Protein expression Biodistribution Percentage Profile (Flux) via IV (intravenous) administration demonstrating organ specific delivery of mRNA to liver, spleen and lung has been achieved via engineered block copolymer lipid hybrid nanoparticles. Other organs include heart, kidney and pancreas; LNP-ON yielded Luciferase protein in liver (98%), while 1.2% in spleen, 0.5% in the lung; BNP-002 produced Luciferase protein in liver (98%), while 0.5% in spleen, 0.3% in the lung; BNP-008 facilitated higher levels of Luciferase protein expression in spleen (67%), liver (26%), 5% in the lung; BNP-012 generated Luciferase protein in spleen (1.4%), liver (0.9%), 92% in the lung; BNP-025 generated Luciferase protein in spleen (13%), liver (2.7%), 76% in the lung.

### DETAILED DESCRIPTION OF THE INVENTION

The following detailed description refers to the accompanying Examples and Figures that show, by way of illustration, specific details and embodiments, in which the invention may be practised. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

A messenger RNA (mRNA) has arisen as a promising strategy for prevention and treatment of various diseases including infections, cancer and gene disorders.

However, the clinical translation of mRNA therapeutic is impeded by its instability and inefficient in vivo delivery. Recently, advanced lipid nanoparticles (LNP) systems have demonstrated potency in preclinical trials and successfully entered the clinics. For instance, LNP-Onpattro (LNP-ON or LNP-ONP) containing DMG-PEG, DSPC, MC3 and Chol (1.5:10.0:50: 38.5) has been approved by the FDA in 2018, where therapeutic siRNA (pastisiran) was encapsulated for treatment of hereditary mediated amyloidosis. To date, many other LNPs have been developed for mRNA delivery. Notably, mRNA-1273 and BNT162b have been used in clinics globally for the prevention of coronavirus disease 2019 (COVID-19).

However, despite the positive results, it remains a challenge to maintain the long-term stability and potency of mRNA loaded LNP. For instance, mRNA-1273 and BNT162b are recommended to be stored at −80° C. and−20° C., respectively. As cold chain transportation and storage are not available in many areas, there is an urgent need to develop therapeutics with enhanced long-term stability.

In the course of the present invention, synthetic polymer-lipid hybrid nanoparticles comprising PBD-PEO (non-degradable) or Poly(ε-caprolactone)-poly(ethylene glycol) (PCL-PEO) (biodegradable) block copolymer, PLGA-PEO have been explored as novel platform for polynucleotide (e.g., mRNA) delivery.

PBD-PEO, PCL-PEO, PLGA-PEO polymer-lipid hybrid nanoparticle were synthesized with well-defined molecular weight and narrow polydispersity. For example, such synthetic polymers can be integrated with helper lipid and ionized lipid and formulated to create a new class of polymer-lipid hybrid nanoparticles (e.g., PBD-PEO polymer lipid hybrid nanoparticles can be interchangeably referred to as “BNPs” herein and PCL-PEO polymer lipid hybrid nanoparticles can be interchangeably referred to as “PCLs” herein) for e.g., mRNA delivery. The effects of compositions and N/P ratios (N in the ionized cationic lipid and P in mRNA) on the performance of the BNPs (prepared by solvent dispersion method) was systematically evaluated in terms of particle size, polydispersity, surface charge, morphology, encapsulation efficiency, loading level and in vitro transfection. The optimal formulation was further produced by Precision Nanosystem Incorporation NanoasembirPatform (PNI). The in vivo delivery efficacy of BNPs and PCLs was further evaluated using Luciferase protein expression model in mice. Importantly, the optimum formulation demonstrated potent mRNA delivery both in vitro and in vivo yet with enhanced storage stability as compared to benchmark LNP-ON. Overall, BNPs demonstrated great potential for delivery of therapeutic mRNA.

In the present context, the term “polynucleotide” (also “nucleic acid”, which can be used interchangeably with the term “polynucleotide”) refers to macromolecules made up of nucleotide units which e.g., can be hydrolysable into certain pyrimidine or purine bases (usually adenine, cytosine, guanine, thymine, uracil), d-ribose or 2-deoxy-d-ribose and phosphoric acid. Non-limiting examples of “polynucleotide” include DNA molecules (e.g. cDNA or genomic DNA), RNA (e.g., siRNA, an mRNA, guide RNA or self-amplifying mRNA (saRNA)), oligonucleotide (e.g., antisense oligonucleotide), combinations thereof or hybrid molecules comprised of DNA and RNA. The nucleic acids can be double- or single-stranded and may contain double- and single-stranded fragments at the same time. Most preferred are double stranded DNA molecules and mRNA molecules.

In the present context, the term “antisense oligonucleotide” refers to a nucleic acid polymer, at least a portion of which is complementary to a nucleic acid which is present in a normal cell or in an affected cell. Exemplary “antisense oligonucleotide” include antisense RNA, SiRNA, RNAi.

In the present context, polymersomes are vesicles with a polymeric membrane, which are typically, but not necessarily, formed from the self-assembly of dilute solutions of one or more amphiphilic block copolymers, which can be of different types such as diblock and triblock (A-B-A or A-B-C). Polymersomes may also be formed of tetra-block or penta-block copolymers. For tri-block copolymers, the central block is often shielded from the environment by its flanking blocks, while di-block copolymers self-assemble into bilayers, placing two hydrophobic blocks tail-to-tail, much to the same effect. In most cases, the vesicular membrane has an insoluble middle layer and soluble outer layers. The driving force for polymersome formation by self-assembly is considered to be the microphase separation of the insoluble blocks, which tend to associate in order to shield themselves from contact with water. Polymersomes possess such properties due to the large molecular weight of the constituent copolymers. Vesicle formation is favored upon an increase in total molecular weight of the block copolymers. As a consequence, diffusion of the (polymeric) amphiphiles in these vesicles is very low compared to vesicles formed by lipids and surfactants. Owing to this less mobility of polymer chains aggregated in vesicle structure, it is possible to obtain stable polymersome morphologies. Unless expressly stated otherwise, the term “polymersome” and “vesicle”, as used herein, are taken to be analogous and may be used interchangeably. Importantly, a polymersome can be formed from either one kind of block copolymers or from two or more kinds of block copolymers, meaning a polymersome can also be formed from mixtures of polymersomes and thus can contain two or more block copolymers.

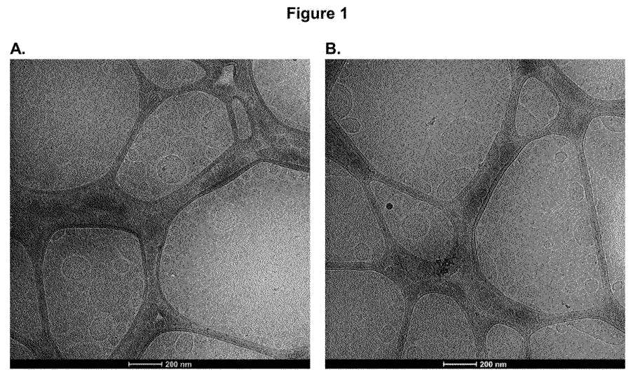

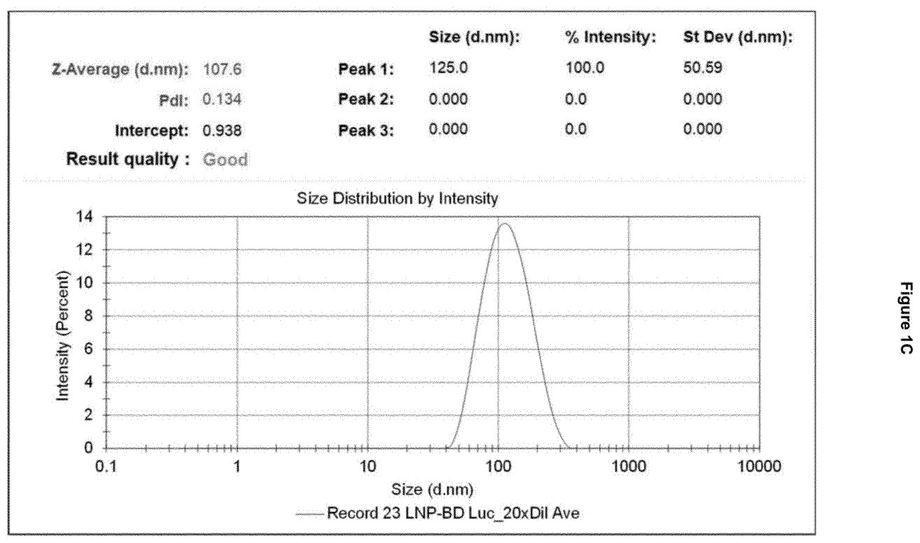

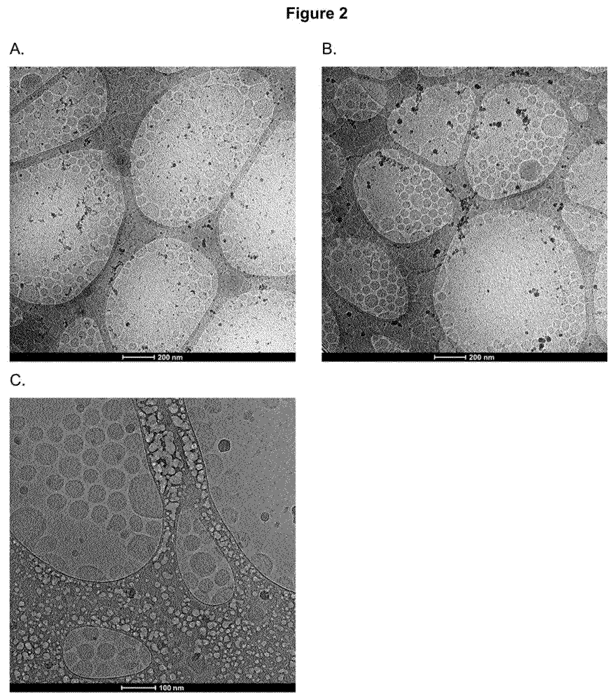

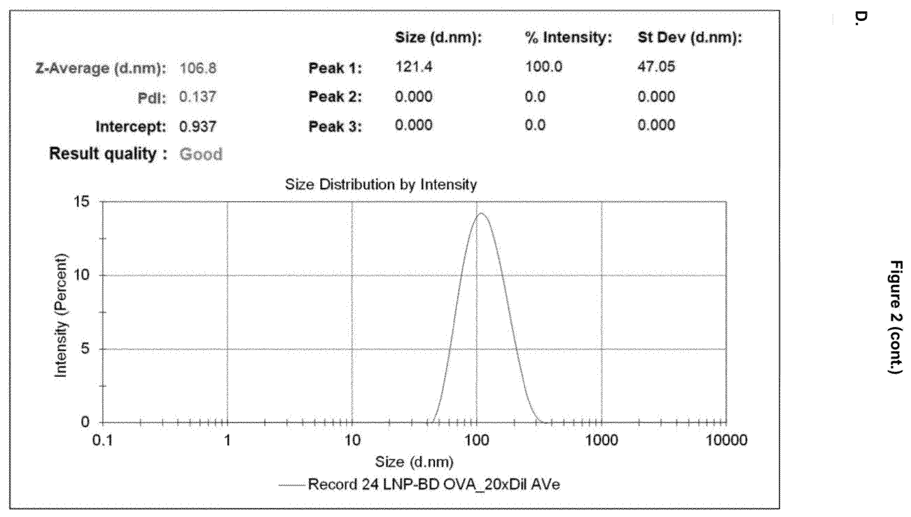

In the present context, polymer-lipid hybrid nanoparticles of the present invention comprising a lipid and a block copolymer, wherein the amount of said lipid, expressed in mole percentage (mole %) present in the polymer-lipid hybrid nanoparticle, wherein the mole percentage refers to the total amount of all components that form the polymer-lipid nanoparticle is greater than the amount of said block copolymer, expressed in mole percentage, present in the polymer-lipid hybrid nanoparticle. Such polymer-lipid hybrid nanoparticles are not polymersomes. They may have electro-lucent amorphous internal structure surrounded by a peripheral bilayer. Exemplary polymer-lipid hybrid nanoparticles of the present invention having one or more of the following characteristics: (i) a diameter greater than 75 nm, e.g., said diameter ranging from about 80 nm to about 450 nm or said diameter ranging from about 80 nm to about 140 nm, or said diameter ranging from about 100 nm to about 140 nm (The diameter can, for example, be determined by a dynamic light scattering (DLS) instrument using Z-average (d, nm), a preferred DLS parameter. Z-average size is the intensity weighted harmonic mean particle diameter (cf. FIGS. 1 and 2)); (ii) a polydispersity index (PDI) greater than about 0.15, e.g., PDI from about 0.175 to about 0.245; (iii) a a zeta potential preferably between-40 mV and +40 mV; (iv) physiochemical properties as shown in one or more of Tables 2, 3, 6A, 6B, FIGS. 1-16 (v) electro-lucent amorphous internal structure surrounded by a peripheral bilayer membrane.

The polymer-lipid hybrid nanoparticle of the present invention, may comprise a soluble encapsulated antigen, wherein said soluble encapsulated antigen is a protein and/or polynucleotide, preferably said protein is a nuclease involved in gene- or RNA-editing, polynucleotide is selected from a RNA (e.g., siRNA, an mRNA, guide RNA or self-amplifying mRNA (saRNA)) molecule or a DNA molecule.

In the present context, the term “encapsulated” means enclosed by a membrane (e.g., membrane of the polymer-lipid hybrid nanoparticle of the present invention, e.g., embodied inside the lumen of said polymer-lipid hybrid nanoparticle). With reference to an antigen the term “encapsulated” further means that said antigen is neither integrated into-nor covalently bound to-nor conjugated to said membrane (e.g., of a polymer-lipid hybrid nanoparticle of the present invention).

In the present context, the term “antigen” means any substance that may be specifically bound by components of the immune system. Only antigens that are capable of eliciting (or evoking or inducing) an immune response are considered immunogenic and are called “immunogens”. Exemplary non-limiting antigens are proteins and polynucleotides. Exemplary non-limiting protein antigen is a nuclease involved in gene- or RNA-editing. Exemplary non-limiting polynucleotide is selected from a RNA (e.g., siRNA, an mRNA (e.g., as set forth in SEQ ID NOs: 1, 2 or 3), guide RNA or self-amplifying mRNA (saRNA) molecule or a DNA molecule. The antigen may originate from within the body (“self-antigen”) or from the external environment (“non-self”).

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

In the present context, the term “CD8 (+) T cell-mediated immune response” refers to the immune response mediated by cytotoxic T cells (also known as TC, cytotoxic T lymphocyte, CTL, T-killer cells, cytolytic T cells, CD8 (+) T-cells or killer T cells). Example of cytotoxic T cells include, but are not limited to antigen-specific effector CD8 (+) T cells. In order for the T-cell receptors (TCR) to bind to the class I MHC molecule, the former must be accompanied by a glycoprotein called CD8, which binds to the constant portion of the class I MHC molecule.

Therefore, these T cells are called CD8 (+) T cells. Once activated, the TC cell undergoes “clonal expansion” with the help of the cytokine Interleukin-2 (IL-2), which is a growth and differentiation factor for T cells. This increases the number of cells specific for the target antigen that can then travel throughout the body in search of antigen-positive somatic cells.

In the present context, the term “cellular immune response” refers to an immune response that does not involve antibodies, but rather involves the activation of phagocytes, antigen-specific cytotoxic T-lymphocytes, and the release of various cytokines in response to an antigen.

In the present context, the term “humoral immune response” refers to an immune response mediated by macromolecules found in extracellular fluids such as secreted antibodies, complement proteins, and certain antimicrobial peptides. Its aspects involving antibodies are often called antibody-mediated immunity.

In the present context, the term “stabilizer” may refer to a substance that renders or maintains a solution, mixture (e.g., polymer-lipid hybrid nanoparticle), suspension or state resistant to chemical change. Exemplary non-limiting stabilizers of the present invention comprise or consist of cholesterol, substituted or unsubstituted cholesterol moiety, or cholesterol derivative, preferably said cholesterol derivative is a hydroxylated cholesterol derivative (e.g., a hydroxycholesterol).

In the present context, the term “B cells”, also known as B lymphocytes, are a type of white blood cell of the lymphocyte subtype. They function in the humoral immunity component of the adaptive immune system by secreting antibodies.

An “antibody” when used herein is a protein comprising one or more polypeptides (comprising one or more binding domains, preferably antigen binding domains) substantially or partially encoded by immunoglobulin genes or fragments of immunoglobulin genes. The term “immunoglobulin” (lg) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., lgG1, lgG2, lgG3, lgG4, IgA1, and IgA2, with IgG being preferred in the context of the present invention. An antibody relating to the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the lgG4 subtype would not have the capability to exert ADCC.

The term “antibody” also includes, but is not limited to, but encompasses monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

The antibody relating to the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

“Polyclonal antibodies” or “polyclonal antisera” refer to immune serum containing a mixture of antibodies specific for one (monovalent or specific antisera) or more (polyvalent antisera) antigens which may be prepared from the blood of animals immunized with the antigen or antigens.

The term “immunizing” refers to the step or steps of administering one or more antigens to a non-human animal so that antibodies can be raised in the animal.

Specifically, the non-human animal is preferably immunized at least two, more preferably three times with said polypeptide (antigen), optionally in admixture with an adjuvant. An “adjuvant” is a nonspecific stimulant of the immune response. The adjuvant may be in the form of a composition comprising either or both of the following components: (a) a substance designed to form a deposit protecting the antigen(s) from rapid catabolisme.g. mineral oil, alum, aluminium hydroxide, liposome or surfactant (e.g. pluronic polyol) and (b) a substance that nonspecifically stimulates the immune response of the immunized host animal (e.g. by increasing lymphokine levels therein).

Exemplary molecules for increasing lymphokine levels include lipopolysaccaride (LPS) or a Lipid A portion thereof; Bordetalla pertussis; pertussis toxin; Mycobacterium tuberculosis; and muramyl dipeptide (MDP). Examples of adjuvants include Freund's adjuvant (optionally comprising killed M. tuberculosis; complete Freund's adjuvant); aluminium hydroxide adjuvant; and monophosphoryl Lipid A-synthetic trehalose dicorynomylcolate (MPL-TDM).

The “non-human animal” to be immunized herein is preferably a rodent. A “rodent” is an animal belonging to the rodentia order of placental mammals. Exemplary rodents include mice, rats, guinea pigs, squirrels, hamsters, ferrets etc, with mice being the preferred rodent for immunizing according to the method herein. Other non-human animals which can be immunized herein include non-human primates such as Old World monkey (e.g. baboon or macaque, including Rhesus monkey and cynomolgus monkey; see U.S. Pat. No. 5,658,570); birds (e.g. chickens); rabbits; goats; sheep; cows; horses; pigs; donkeys; dogs etc.

By “screening” is meant subjecting one or more monoclonal antibodies (e.g., purified antibody and/or hybridoma culture supernatant comprising the antibody) to one or more assays which determine qualitatively and/or quantitatively the ability of an antibody to bind to an antigen of interest.

By “immuno-assay” is meant an assay that determines binding of an antibody to an antigen, wherein either the antibody or antigen, or both, are optionally adsorbed on a solid phase (i.e., an “immunoadsorbent” assay) at some stage of the assay. Exemplary such assays include ELISAs, radioimmunoassays (RIAs), and FACS assays. Given the above, the present invention provides thus a monoclonal or polyclonal antibody obtainable by the aforedescribed methods for the generation of an antibody, i.e., by immunizing a non-human animal as described before.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

Non-limiting examples of cancers include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of luekemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (MI), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation, lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary, plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 or PD-L1 antibody), and recurrent cancers.

The term “subject” is intended to include living organisms. Examples of subjects include mammals, e.g., humans, dogs, cows, horses, pigs, sheep, goats, cats, mice, rabbits, rats, and transgenic non-human animals. The subject (animal) can however be a non-mammalian animal such as a bird or a fish. In some preferred embodiments of the invention, the subject is a human, while in other some other preferred embodiments, the subject might be a farm animal, wherein the farm animal can be either a mammal or a non-mammalian animal. Examples of such non-mammalian animals are birds (e.g. poultry such as chicken, duck, goose or turkey), fishes (for example, fishes cultivated in aquaculture such as salmon, trout, or tilapia) or crustacean (such as shrimps or prawns). Examples of mammalian (life stock) animals includes goats; sheep; cows; horses; pigs; or donkeys. Other mammals include cats, dogs, mice and rabbits, for example. In illustrative embodiments the polymer-lipid hybrid nanoparticles of the present invention are used for the vaccination or immunization of the above-mentioned farm animals, both mammalian farm animals and non-mammalian farm animals (a bird, a fish, a crustacean) against virus infections (cf. the Example section in this regard). Accordingly, in such cases, polymer-lipid hybrid nanoparticle of the invention may have encapsulated therein soluble viral full length proteins or soluble fragments of viral full-length proteins.

When used for vaccinations of both humans and non-humans animals, polymer-lipid hybrid nanoparticle or compositions comprising polymer-lipid hybrid nanoparticle of the invention may be administered orally to the respective subject (cf. also the Example Section) dissolved only in a suitable (pharmaceutically acceptable) buffer such as phosphate-buffered saline (PBS) or 0.9% saline solution (an isotonic solution of 0.90% w/v of NaCl, with an osmolality of 308 mOsm/L).

As used herein, the term “LNP-Onpattro”, which can be used interchangeably with the terms “LNP-ON” or “LNP-ONP” may refer to lipid nanoparticles containing DMG-PEG, DSPC, MC3 and Chol, e.g., at a mole ratio 1.5:10.0:50: 38.5.

In illustrative embodiments of these polymer-lipid hybrid nanoparticles and oral formulations, the polymer-lipid hybrid nanoparticles that are used for vaccination have encapsulated therein a viral antigen that comprises a soluble portion of Influenza hemagglutinin, Swine Influenza hemagglutinin, Foot and Mouth Disease (FMD) virus protein such as the VP1, VP2 or VP3 coat protein (the VP1 coat protein contains the main antigenic determinants of the FMD virion, and hence changes in its sequence should be responsible for the high antigenic variability of the virus), Ovalbumin (OVA) or of the Porcine epidemic diarrhea (PED) virus SPIKE protein.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the subject's own immune system. The term “patient” includes human and other mammalian subjects that receive either prophylactic or therapeutic treatment.

The appropriate dosage, or therapeutically effective amount, of the antibody or antigen binding portion thereof will depend on the condition to be treated, the severity of the condition, prior therapy, and the patient's clinical history and response to the therapeutic agent. The proper dose can be adjusted according to the judgment of the attending physician such that it can be administered to the patient one time or over a series of administrations. The pharmaceutical composition can be administered as a sole therapeutic or in combination with additional therapies as needed.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

Pharmaceutical compositions for injection may be presented in unit dosage form, e.g., in ampoules or in multi-dose containers, with an added preservative. In addition, a number of recent drug delivery approaches have been developed and the pharmaceutical compositions of the present invention are suitable for administration using these new methods, e.g., Inject-ease, Genject, injector pens such as Genen, and needleless devices such as MediJector and BioJector. The present pharmaceutical composition can also be adapted for yet to be discovered administration methods. See also Langer, 1990, Science, 249:1527-1533.

The pharmaceutical composition can also be formulated as a depot preparation. Such long acting formulations may be administered by implantation (for example subcutaneously, into the ligament or tendon, subsynovially or intramuscularly), by subsynovial injection or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may also be in a variety of conventional depot forms employed for administration to provide reactive compositions. These include, for example, solid, semi-solid and liquid dosage forms, such as liquid solutions or suspensions, slurries, gels, creams, balms, emulsions, lotions, powders, sprays, foams, pastes, ointments, salves, balms and drops.

The pharmaceutical compositions may, if desired, be presented in a vial, pack or dispenser device which may contain one or more unit dosage forms containing the active ingredient. In one embodiment, the dispenser device can comprise a syringe having a single dose of the liquid formulation ready for injection. The syringe can be accompanied by instructions for administration.

The formulations described herein are useful as pharmaceutical compositions in the treatment and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

As used herein, the term “treating” and “treatment” refers to administering to a subject a therapeutically effective amount of a pharmaceutical composition according to the invention. A “therapeutically effective amount” refers to an amount of the pharmaceutical composition or the antibody which is sufficient to treat or ameliorate a disease or disorder, to delay the onset of a disease or to provide any therapeutic benefit in the treatment or management of a disease.

As used herein, the term “prophylaxis” refers to the use of an agent for the prevention of the onset of a disease or disorder. A “prophylactically effective amount” defines an amount of the active component or pharmaceutical agent sufficient to prevent the onset or recurrence of a disease.

As used herein, the terms “disorder” and “disease” are used interchangeably to refer to a condition in a subject. In particular, the term “cancer” is used interchangeably with the term “tumor”.

The kit of the invention will typically comprise the container described above and one or more other containers comprising materials desirable from a commercial and user standpoint, including buffers, diluents, filters, needles, syringes, and package inserts with instructions for use.

In the present context, the term “soluble antigen” as used herein means an antigen capable of being dissolved or liquefied. The term “soluble antigen” includes antigens that were “solubilized”, i.e., rendered soluble or more soluble, especially in water, by the action of a detergent or other agent. Exemplary non-limiting soluble antigens of the present invention include: polypeptides derived from a non-soluble portion of proteins, hydrophobic polypeptides rendered soluble for encapsulation as well as aggregated polypeptides that are soluble as aggregates. In some aspects, the antigens (e.g., membrane proteins) of the present invention are solubilized with the aid of detergents, surfactants, temperature change or pH change.

In some aspects, the invention provides a polymer-lipid hybrid nanoparticle comprising a lipid and a block copolymer, wherein the amount of said lipid, expressed in mole percentage (i.e., a mole %) present in the polymer-lipid hybrid nanoparticle, wherein the mole percentage refers to the total amount of all components that form the polymer-lipid nanoparticle, is greater than the amount of said block copolymer, expressed in mole percentage, present in the polymer-lipid hybrid nanoparticle.

In some aspects, the invention provides a polymer-lipid hybrid nanoparticle as described herein, wherein the lipid (e.g., ionizable lipid) is selected from a group consisting of: an ionizable lipid DLin-MC3-DMA (also referred to as MC3) and an ionizable lipid C12-200.

In some aspects, the invention provides a polymer-lipid hybrid nanoparticle as described herein, wherein the block copolymer is selected from a group consisting of: PBD-PEO block copolymer, PCL-PEO block copolymer and DMG-PEGblock copolymer (e.g., Table 1).

TABLE 1
Exemplary polymers & lipids used in polymer-lipid hybrid nanoparticles of the present invention:

| Name | Structure | Charge | MW |
|---|---|---|---|
| Cholesterol | 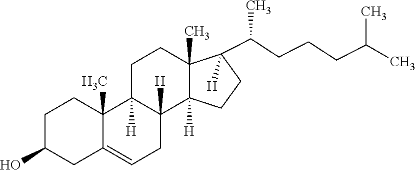 | Neutral | 386.7 |
| 1,2-Dimyristoyl-sn-glycero-3- phosphocholine (DMPC, 14:0 PC) | 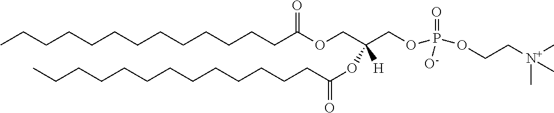 [SMILES: CCCCCCCCCCCCCC(=O)OC[C@H](COP(=O)([O-])OCC[N+](C)(C)C)OC(=O)CCCCCCCCCCCCC] | Neutral | 678.0 |
| 1,2-Distearoyl-sn-glycero-3- phosphocholine (DSPC, 18:0 PC) | 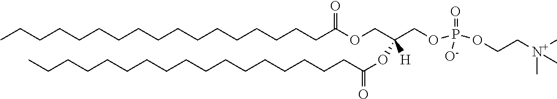 [SMILES: CCCCCCCCCCCCCCCCCC(=O)OC[C@H](COP(=O)([O-])OCC[N+](C)(C)C)OC(=O)CCCCCCCCCCCCCCCCC] | Neutral | 790.1 |
| 1,2-dioleoyl-sn-glycero-3- phosphoethanolamine (DOPE) | 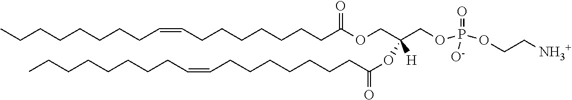 [SMILES: CCCCCCCC/C=C\CCCCCCCC(=O)OC[C@H](COP(=O)([O-])OCC[NH3+])OC(=O)CCCCCCC/C=C\CCCCCCCC] | Neutral | 744.0 |
| DLin-MC3-DMA | 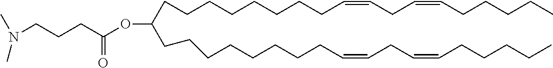 [SMILES: CCCCC/C=C\C/C=C\CCCCCCCCC(CCCCCCCC/C=C\C/C=C\CCCCC)OC(=O)CCCN(C)C] | Positive (pH <6.4) | 642.1 |
| DOTAP | 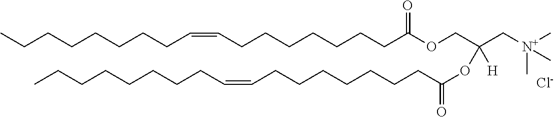 [SMILES: CCCCCCCC/C=C\CCCCCCCC(=O)OCC(C[N+](C)(C)C)OC(=O)CCCCCCC/C=C\CCCCCCCC ; [Cl-]] | Positive | 698.5 |
| DMG-PEG-2K | 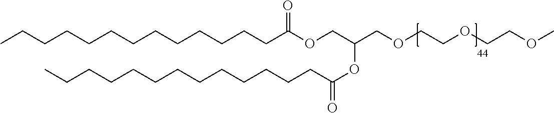 | Neutral | 2509.2 |
| PBD_{1.2k}-b-PEO_{0.6k} | 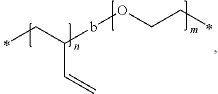 | Neutral | 1700- 2100 |
| | wherein n = 22; b denotes block, m = 12; * = OCH₃ | | |
| PCL_{3.3k}-b-PEO₁ₖ | 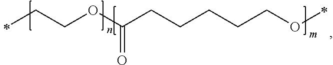 | Neutral | 4300 |
| | wherein n = 22; m = 29; * = H | | |
| PLGA_{1.9k}-b-PEO₁ₖ | 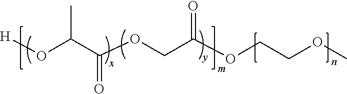 | Neutral | 2860 |
| | wherein x = 23; y = 4; m = 26; n = 29 | | |

In some aspects, the invention provides a polymer-lipid hybrid nanoparticle as described herein, wherein a mole % ratio of the lipid to the block copolymer is between 31.8 to 12 and about 35 to 2.5.

In some aspects, the invention provides a polymer-lipid hybrid nanoparticle as described herein, further comprising a stabilizer, e.g., comprising or consisting of cholesterol (also referred to as CHOL).

In some aspects, the invention provides a polymer-lipid hybrid nanoparticle as described herein, further comprising another lipid, wherein said another lipid is selected from a group consisting of: DMPC, DSPC, DOPE, DOTAP, DODAP, DOTMA, DODMA, DDA, 18:1 PA (1,2-dioleoyl-sn-glycero-3-phosphate), 14:0 PA (1,2-dimyristoyl-sn-glycero-3-phosphate), 18:1 BMP (bis(monooleoylglycero)phosphate) (e.g., Table 1).

In some aspects, the invention provides a polymer-lipid hybrid nanoparticle as described herein, consisting of: (i) PBD-PEO, MC3, CHOL; (ii) PBD-PEO, C12-200, CHOL; (iii) PBD-PEO, DOPE, C12-200, CHOL; (iv) PBD-PEO, DOPE, C12-200, CHOL; (v) PBD-PEO, DOPE, C12-200, CHOL; (vi) PBD-PEO, DOPE, C12-200, CHOL; (vii) DMG-PEG, DSPC, MC3, CHOL; (viii) PCL-PEO, DMPC, MC3, CHOL; (ix) PCL-PEO, DMPC, MC3, CHOL; (x) PCL-PEO, DMPC, MC3, CHOL; or (xi) PCL-PEO, DMPC, MC3, CHOL; (xii) PLGA-PEO, DMPC, MC3, CHOL.

In some aspects, the invention provides a polymer-lipid hybrid nanoparticle as described herein, further comprising a soluble encapsulated antigen, wherein said soluble encapsulated antigen is a protein and/or polynucleotide.

In some aspects, the invention provides a polymer-lipid hybrid nanoparticle as described herein, capable of maintaining long-term stability and/or potency of said polynucleotide (e.g., mRNA, e.g., as set forth in SEQ ID NOs: 1, 2 or 3).

In some aspects, the invention provides a composition comprising a polymer-lipid hybrid nanoparticle as described herein.

In some aspects, the invention provides a method of delivering nucleotide/s to inside a cell without using viral vector/s as delivery means, said method comprising: (i) providing the polymer-lipid hybrid nanoparticle and/or composition of the present invention; and (ii) contacting said polymer-lipid hybrid nanoparticle and/or composition with a cell.

In some aspects of the present invention, a polymer-lipid hybrid nanoparticle of the present invention is selected from the group consisting of: (a) BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2); (b) BNP-002 having 5 mM (Molar %) of DLin-MC3-DMA: Cholesterol: PBD-b-PEO (49:39:12); or (c) BNP-002.2 having 5 mM (Molar %) of DLin-MC3-DMA: Cholesterol: DSPC: PBD-b-PEO (49.3:39.0:10.1:1.6).

In some aspects of the present invention, a polymer-lipid hybrid nanoparticle of the present invention is capable of targeting (e.g., predominantly targeting) a tissue/s and/or cell/s of an organ selected from the group consisting of: liver, spleen, lung/s, preferably said targeting is carried out without using a functional ligand/s; further preferably wherein: (a) the following polymer-lipid hybrid nanoparticles are suitable (e.g., is used) for said lung targeting: BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2); (b) the following polymer-lipid hybrid nanoparticle/s are suitable (e.g., is used) for said liver targeting: BNP-002 having 5 mM (Molar %) of DLin-MC3-DMA: Cholesterol: PBD-b-PEO (49:39:12); (c) the following polymer-lipid hybrid nanoparticle/s are suitable (e.g., is used) for said spleen targeting: BNP-002.2 having 5 mM (Molar %) of DLin-MC3-DMA: Cholesterol: DSPC: PBD-b-PEO (49.3:39.0:10.1:1.6).

Based on the above, a new class of lipid hybrid nanoparticles has been developed in the course of the present invention, which is particularly suitable for mRNA delivery. Illustrative optimal polymer-lipid hybrid nanoparticles of the present invention exhibite favorable physicochemical properties and/or superior encapsulation efficiency (˜100%). In comparison to benchmark LNP-ON, the optimal formulation of the polymer-lipid hybrid nanoparticle of the present invention out perform with enhance in vitro transfection efficacy and/or long term thermostability, as can be evidenced by high levels of Luc protein expression and OVA protein expression (e.g., see below in the Experimental Section). Moreover, the ACM polymer-lipid hybrid nanoparticle formulations display less cytotoxicity as compared to benchmark LNP-ON (e.g., see below in the Experimental Section). Importantly, the optimal formulation of the polymer-lipid hybrid nanoparticle of the present invention demonstrate potent in vivo mRNA delivery efficacy, which is comparable to that of benchmark LNP-ON. Furthermore, OVA mRNA formulation can strongly activate cDC1 and cDC2 in the lymph nodes to promote antigen surface presentation. Taken together, the present invention provides a novel class of polymer lipid hybrid nanoparticles with efficient protein and antigen expression as well as enhanced thermostability, which hold great potential for delivery of therapeutic mRNA over a wide range of diseases.

The invention is also characterized by the following items:
1. A polymer-lipid hybrid nanoparticle comprising an lipid and a block copolymer, wherein the amount of said lipid, expressed in mole percentage (mole %) present in the polymer-lipid hybrid nanoparticle, wherein the mole percentage refers to the total amount of all components that form the polymer-lipid nanoparticle is greater than the amount of said block copolymer, expressed in mole percentage, present in the polymer-lipid hybrid nanoparticle, preferably said greater is at least about two-fold greater, further preferably said greater is at least about 3-fold greater, most preferably said greater is at least about 4-fold greater, further most preferably said greater is at least about 5-fold greater.
2. The polymer-lipid hybrid nanoparticle according to any one of preceding items, wherein a mole % ratio of said lipid to said block copolymer is from about 31.8:12 to about 35:2.5, preferably said mole % ratio is selected from the group consisting of:
   a) 49:12;
   b) 35:2.5;
   c) 31.8:12;
   d) 35:12;
   e) 23.8:4.8;
   f) 45:10;
   g) 49:8.
3. The polymer-lipid hybrid nanoparticle according to any one of preceding items, wherein said polymer-lipid hybrid nanoparticle having one or more of the following characteristics:
   a) said polymer-lipid hybrid nanoparticle is synthetic;
   b) a diameter greater than 75 nm, preferably said diameter ranging from about 80 nm to about 450 nm, further preferably said diameter ranging from about 80 nm to about 140 nm, most preferably said diameter ranging from about 100 nm to about 140 nm;
   c) a polydispersity index (PDI) greater than about 0.15, preferably greater than about 0.17, further preferably PDI from about 0.175 to about 0.245; and/or
   d) a zeta potential from about-40 mV to about +40 mV, preferably said zeta potential is greater than 12 mV;
   e) are spherical particles with amorphous structure;
   f) having multi-compartmental structure;
   g) are vesicles with heterogeneous structure (e.g., capable of fusion and budding) surrounded by a bilayer.
4. The polymer-lipid hybrid nanoparticle according to any one of preceding items, wherein polymer-lipid hybrid nanoparticle further comprising a stabilizer, preferably said stabilizer comprising cholesterol (or CHOL, e.g., having Formula I:

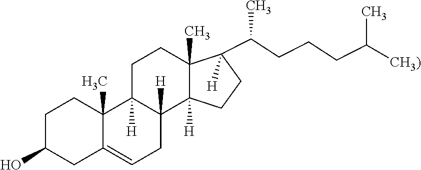

and/or substituted or unsubstituted cholesterol moiety.
5. The polymer-lipid hybrid nanoparticle according to any one of preceding items, wherein said stabilizer is selected from the group consisting of: cholesterol, substituted or unsubstituted cholesterol moiety, cholesterol derivative, preferably said cholesterol derivative is a hydroxylated cholesterol derivative (e.g., a hydroxycholesterol).
6. The polymer-lipid hybrid nanoparticle according to any one of preceding items, further comprising another lipid, preferably said lipid is cationic, ionizable cationic lipid and/or anionic lipid selected from a group consisting of:
   a) 1,2-Dimyristoyl-sn-glycero-3-phosphocholine (DMPC, 14:0 PC), e.g., having Formula II:

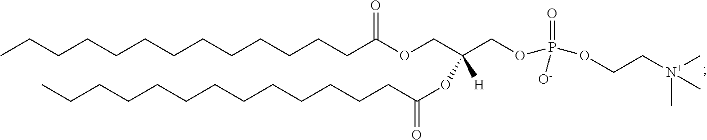 [SMILES: CCCCCCCCCCCCCC(=O)OC[C@H](COP(=O)([O-])OCC[N+](C)(C)C)OC(=O)CCCCCCCCCCCCC]

b) 1,2-Distearoyl-sn-glycero-3-phosphocholine (DSPC, 18:0 PC), e.g., having Formula III:

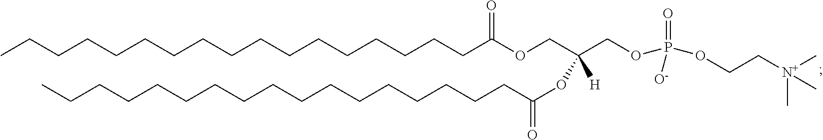 [SMILES: CCCCCCCCCCCCCCCCCC(=O)OC[C@H](COP(=O)([O-])OCC[N+](C)(C)C)OC(=O)CCCCCCCCCCCCCCCCC]

c) 1,2-dioleoyl-sn-glycero-3-phosphoethanolamine (DOPE), e.g., having Formula IV:

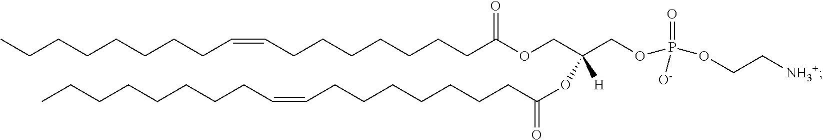

d) 1,2-dioleoyl-3-trimethylammonium-propane (DOTAP), e.g., having Formula V:

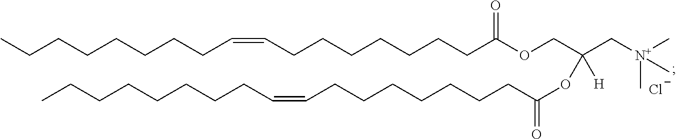 [SMILES: CCCCCCCC/C=C\CCCCCCCC(=O)OCC(C[N+](C)(C)C)OC(=O)CCCCCCC/C=C\CCCCCCCC ; [Cl-]]

e) 1,2-Dioleoyl-3-trimethylammonium propane (DODAP), e.g., having Formula VI:

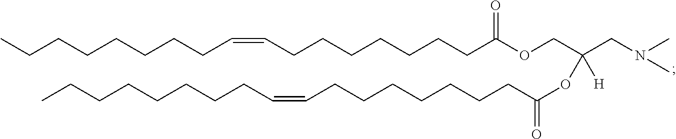 [SMILES: CCCCCCCC/C=C\CCCCCCCC(=O)OCC(CN(C)C)OC(=O)CCCCCCC/C=C\CCCCCCCC]

f) 1,2-di-O-octadecenyl-3-trimethylammonium propane (DOTMA), e.g., having Formula VII:

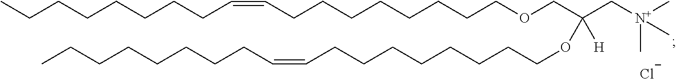 [SMILES: CCCCCCCC/C=C\CCCCCCCCOCC(C[N+](C)(C)C)OCCCCCCCC/C=C\CCCCCCCC ; [Cl-]]

g) 1,2-dioleyloxy-3-dimethylaminopropane (DODMA), e.g., having Formula VIII:

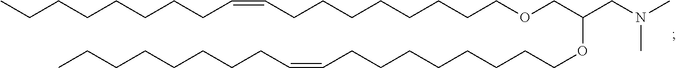 [SMILES: CCCCCCCC/C=C\CCCCCCCCOCC(CN(C)C)OCCCCCCCC/C=C\CCCCCCCC]

h) Dimethyldioctadecylammonium (DDA), e.g. having Formula IX:

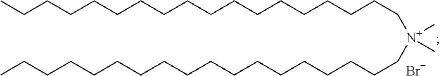 [SMILES: CCCCCCCCCCCCCCCCCC[N+](C)(C)CCCCCCCCCCCCCCCCCC ; [Br-]]

i) 1,2-dioleoyl-sn-glycero-3-phosphate (18:1 PA), e.g. having Formula X:

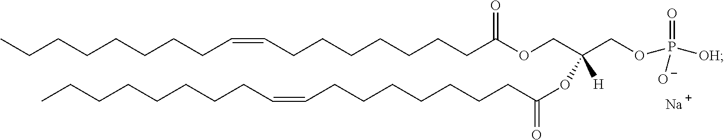 [SMILES: CCCCCCCC/C=C\CCCCCCCC(=O)OC[C@H](COP(=O)([O-])O)OC(=O)CCCCCCC/C=C\CCCCCCCC ; [Na+]]

j) 1,2-dimyristoyl-sn-glycero-3-phosphate (14:0 PA), e.g. having Formula XI:

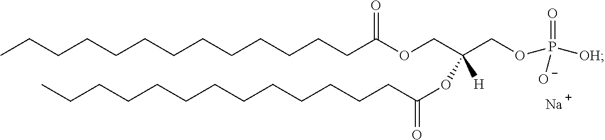 [SMILES: CCCCCCCCCCCCCC(=O)OC[C@H](COP(=O)([O-])O)OC(=O)CCCCCCCCCCCCC ; [Na+]]

k) bis(monooleoyiglycero)phosphate (e.g., S and/or R isomer) (18:1 BMP), e.g., having Formula XII:

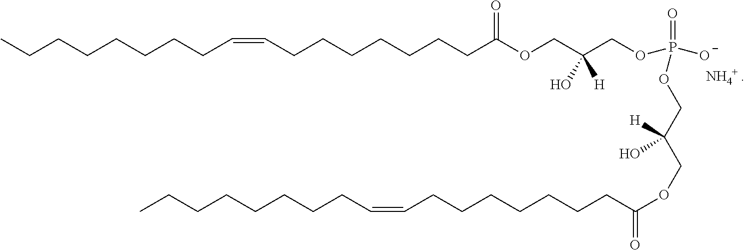

7. The polymer-lipid hybrid nanoparticle according to any one of preceding items, wherein said lipid (e.g., ionizable lipid) is selected from a group consisting of:
   a) an ionizable lipid DLin-MC3-DMA (or MC3, i.e., (6Z,9Z,28Z,31Z)-Heptatriaconta-6,9,28,31-tetraen-19-yl 4-(dimethylamino) butanoate) having Formula XIII:

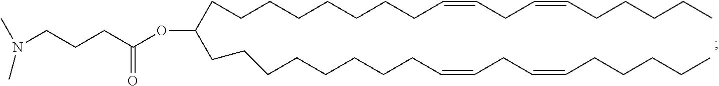 [SMILES: CCCCC/C=C\C/C=C\CCCCCCCCC(CCCCCCCC/C=C\C/C=C\CCCCC)OC(=O)CCCN(C)C]

b) an ionizable lipid C12-200, e.g., having Formula XIV:

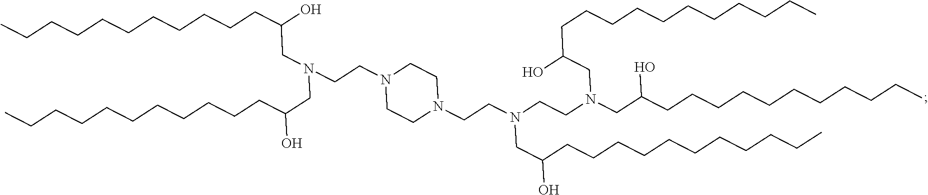 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

c) an ionizable lipid 3060/10, e.g., having Formula XVIII (e.g., CAS Number 2322290-93-5):

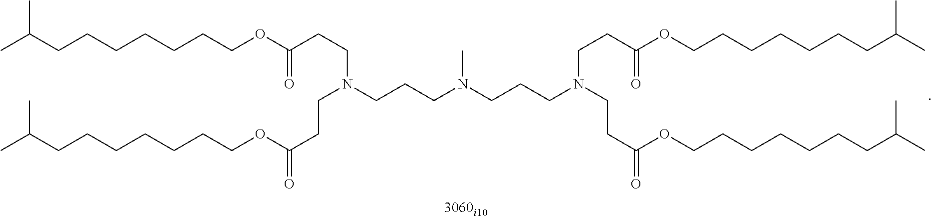 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C]

8. The polymer-lipid hybrid nanoparticle according to any one of preceding items, wherein said block copolymer is selected from a group consisting of:
   a) Poly(butadiene)-poly(ethylene oxide) (PB-PEO) diblock copolymer (e.g., PB-PEO diblock copolymer comprises 5-50 blocks PB and 5-50 blocks PEO);
   b) Poly(dimethylsiloxane)-poly(ethylene oxide) (PDMS-PEO) diblock copolymer (e.g., linear having Formula XVII:

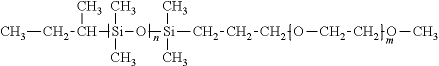

or
comb-type having Formula XVIII:

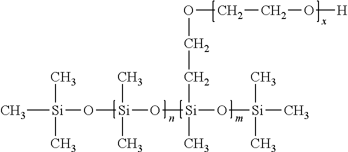

c) poly(dimethyl siloxane)-poly(acrylic acid) (PDMS-PAA);
d) PBD-PEO block copolymer, wherein said PBD-PEO diblock copolymer comprises 5-50 blocks PBD and 5-50 blocks PEO e.g., PBD_{1.2k}-b-PEO_{0.6k}, wherein k=1000 Da, e.g., having Formula XV:

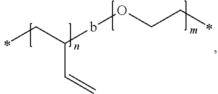

wherein n=22; b denotes block, m=12; *=OCH3;
e) PCL-PEO block copolymer, wherein said PCL-PEO diblock copolymer comprises 5-50 blocks PCL and 5-50 blocks PEO e.g., PCL_{3.3k}-b-PEO₁ₖ, k=1000 Da, e.g., having Formula XVI:

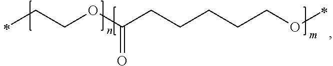

wherein n=22; m=29; *=H;
f) PLGA-PEO block copolymer, wherein said PLGA-PEO di-block copolymer comprises 5-50 blocks PLGA and 5-50 blocks PEO e.g., PLGA_{1.9k}-b-PEO₁ₖ, k=1000 Da, e.g., having Formula XVI:

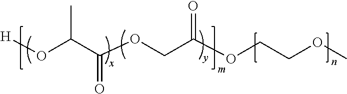

wherein x=23; y=4; m=26; n=29
g) PLA-PEO block copolymer, wherein said PLA-PEO di-block copolymer comprises 5-50 blocks PLA and 5-50 blocks PEO;
h) PDMS-PEO (poly(dimethylsiloxane)-poly(ethylene oxide) block copolymer or the triblock copolymer PMOXA-PDMS-PMOXA (poly(2-methyloxazoline)-poly (dimethylsiloxane)-poly(2-methyloxazoline), where in PDMS copolymer comprises 5-60 blocks of PDMS, 5-50 blocks PEO, and 5-50 blocks of PMOXA;
i) PVP-PLA (polyvinylpyrrolidone-polylactic acid) block copolymer;
j) poly(N-vinylpyrrolidone-b-Polylactic acid;
k) PIB-PAA (Polyisobutylene-Polyacrylic acid);
l) PIP-PEO (polyisoprene-Polyethylene oxide);
m) 1,2-dimyristoyl-rac-glycero-3-methoxypolyethylene glycol-2000 (i.e., DMG-PEGblock copolymer, e.g., DMG-PEG2, wherein k=1000 Da), e.g., having Formula XVII:

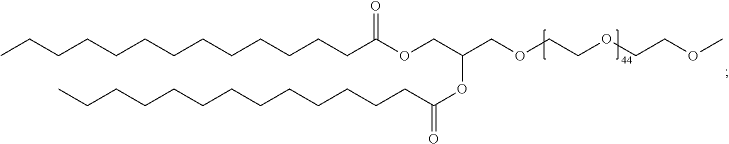

n) poly(ε-caprolactone-co-δ-valerolactone) P(CL-VL)-PEG diblock copolymer.
9. The polymer-lipid hybrid nanoparticle according to item 8, wherein said block copolymer is PBD-b-PEO or PCL-PEO.
10. The polymer-lipid hybrid nanoparticle according to any one of preceding items, wherein said polymer-lipid hybrid nanoparticle comprising or consisting of:
   a) PBD-PEO, MC3, CHOL, preferably at about a following mole ratio: 12: 49:39;
   b) PBD-PEO, C12-200, CHOL, preferably at about a following mole ratio: 12: 49:39;
   c) PBD-PEO, DOPE, C12-200, CHOL, preferably at about a following mole ratio: 2.5:16: 35:46.5;
   d) PBD-PEO, DOPE, C12-200, CHOL, preferably at about a following mole ratio: 12: 12.8:31.8:43.4;
   e) PBD-PEO, DOPE, C12-200, CHOL, preferably at about a following mole ratio: 12: 6.5:35: 46.5;
   f) PBD-PEO, DOPE, C12-200, CHOL, preferably at about a following mole ratio: 4.8:23. 8:23.8:47.6;
   g) DMG-PEG, DSPC, MC3, CHOL, preferably at about a following mole ratio: 1.6:10. 1:49.3:39.0;
   h) PCL-PEO, DMPC, MC3, CHOL, preferably at about a following mole ratio: 10: 10:45.0:35.0;
   i) PCL-PEO, DMPC, MC3, CHOL, preferably at about a following mole ratio: 10: 10:45.0:35.0;
   j) PCL-PEO, DMPC, MC3, CHOL, preferably at about a following mole ratio: 8: 4:49.0:39.0;
   k) PCL-PEO, DMPC, MC3, CHOL, preferably at about a following mole ratio: 8: 4:49.0:39.0;
   l) DLin-MC3-DMA, Cholesterol, PBD-b-PEO, preferably at about a following mole ratio: 49:39:12;
   m) DLin-MC3-DMA, Cholesterol, DSPC: PBD-b-PEO, preferably at about a following mole ratio: 49.3:39.0:10.1:1.6;
   n) DOTAP, Cholesterol, DSPC: PBD-b-PEO, preferably at about a following mole ratio: 40:48:10:2;
   o) DOTMA, Cholesterol: DSPC, PBD-b-PEO, preferably at about a following mole ratio: 40:48:10:2.
11. The polymer-lipid hybrid nanoparticle according to any one of preceding items, further comprising a soluble encapsulated antigen, wherein said soluble encapsulated antigen is a protein and/or polynucleotide, preferably said protein is a nuclease involved in gene- or RNA-editing, polynucleotide is selected from a RNA (e.g., siRNA, an mRNA (e.g., SEQ ID NO: 1, SEQ ID NO: 2 or SEQ ID NO: 3), guide RNA or self-amplifying mRNA (saRNA) or antisense oligonucleotide) molecule or a DNA molecule.
12. The polymer-lipid hybrid nanoparticle according to any one of preceding items, capable of maintaining long-term stability and/or potency of said polynucleotide (e.g., siRNA, an mRNA, guide RNA or self-amplifying mRNA (saRNA)), preferably as compared to as compared to LNP-ON.
13. The polymer-lipid hybrid nanoparticle according to any one of preceding items, wherein said nanoparticle is capable and/or characterized of/by one or more of the following:
   a) characterized as shown in one or more of Tables 1, 2, 3, 4, 5, 6A and/or 6B, Table 7, 8, 9, 10 and/or 11 herein;
   b) electro-lucent amorphous internal structure surrounded by a peripheral bilayer;
   c) expressing said polynucleotide (e.g., 6 or 24 hours post-transfection), preferably said polynucleotide is selected from a RNA (e.g., an mRNA or self-amplifying mRNA (saRNA) or antisense oligonucleotide) molecule or a DNA molecule;
   d) eliciting a cellular and/or humoral immune response;
   e) eliciting an immune response, whereinsaid immune response comprising (i) activating dendritic cells (DCs) (e.g., in lymph nodes) and/or (ii) surface presentation of a polypeptide encoded by said polynucleotide;
   f) eliciting a CD8 (+) T cell-mediated immune response, preferably said eliciting is an in vivo, ex vivo or in vitro eliciting;
   g) eliciting a CD4 (*) T cell-mediated immune response, preferably said eliciting is an in vivo, ex vivo or in vitro eliciting;
   h) eliciting a humoral immune response comprising production of specific antibodies (e.g., against a polypeptide encoded by said polynucleotide), further preferably said humoral immune response is an in vivo, ex vivo or in vitro immune response;
   i) capable of predominantly (e.g., at least 51%, e.g., at least 55%, at least 60%, at least 65%, least 70%, least 75%, least 80%, least 85%, least 90%, least 95%, least 90%, least 95%, least 98% or least 99%) targeting spleen (e.g., at 6 h post IV injection) (e.g., BNP-008, e.g., polymer-lipid hybrid nanoparticle having a following mole ratio of PBD-PEO: DOPE: C12-200: CHOL 12:6.5:35: 46.5);
   j) capable of predominantly (e.g., at least 51%, e.g., at least 55%, at least 60%, at least 65%, least 70%, least 75%, least 80%, least 85%, least 90%, least 95%, least 90%, least 95%, least 98% or least 99%) targeting liver (e.g., at 6 h post IV injection), preferably said polymer-lipid hybrid nanoparticle is BNP-002 having 5 mM (Molar %) of DLin-MC3-DMA: Cholesterol: PBD-b-PEO (49:39:12); further preferably said polymer-lipid hybrid nanoparticle is PCL-008 (e.g., as described herein, e.g., consisting of PCL-PEO: DMPC: MC3: CHOL with mole ratio 10:10: 45.0:35.0) and/or PCL-012 (e.g., as described herein, e.g., consisting of PCL-PEO: DMPC: MC3: CHOL with mole ratio 8:4:49.0:39.0);
   k) capable of predominantly (e.g., at least 51%, e.g., least 76%, least 80%, least 85%, least 90%, least 95%, least 90%, least 95%, least 98% or least 99%) targeting lungs (e.g., at 6 h post IV injection), preferably said polymer-lipid hybrid nanoparticle is BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2).
14. The polymer-lipid hybrid nanoparticle according to any one of preceding items, wherein said nanoparticle is capable of targeting antigen presenting cells, wherein said nanoparticle is not attached to a ligand (e.g., an antibody) capable of targeting and/or binding to said antigen presenting cells.
15. The polymer-lipid hybrid nanoparticle according to any one of preceding items, wherein said nanoparticle is capable of targeting cells, wherein said nanoparticle is attached to a ligand (e.g., an antibody) capable of targeting and/or binding to said target cells.
16. The polymer-lipid hybrid nanoparticle according to any one of preceding items, wherein said nanoparticle is capable of selectively targeting tissues and/or organs (e.g., liver, spleen, lungs and/or kidney), wherein said nanoparticle is not functionalized or attach to any ligand (e.g., N-Acetylgalactosamine (GalNac), antibody) capable of targeting and/or binding to said target tissues and organs.
17. The polymer-lipid hybrid nanoparticle according to any one of preceding items, wherein said nanoparticle is capable of selectively targeting tissues and/or organs (e.g., liver, spleen, lungs and/or kidney), wherein said nanoparticle is functionalized or attach to any ligand (e.g., N-Acetylgalactosamine (GalNac), antibody) capable of targeting and/or binding to said target tissues and organs.
18. The polymer-lipid hybrid nanoparticle according to any one of preceding items obtainable by a solvent dispersion method or a micro-mixing technique/s (e.g., as described in the Experimental Section herein, e.g., wherein the N/P ratio (N in the ionized cationic lipid and P in mRNA) is in the range from about 4 to about 40).
19. The polymer-lipid hybrid nanoparticle according to any one of preceding items, wherein said polymer-lipid hybrid nanoparticle is not a polymersome.
20. The polymer-lipid hybrid nanoparticle according to any one of preceding items, wherein said polymer-lipid hybrid nanoparticle is capable of targeting (e.g., predominantly targeting, e.g., at least 51%) a tissue/s and/or cell/s of an organ selected from the group consisting of:
   a) liver,
   b) spleen,
   c) lung/s,
   preferably said targeting is carried out without using a functional ligand/s;
   further preferably wherein:
      a) the following polymer-lipid hybrid nanoparticles are suitable for said lung targeting: BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2);
      b) the following polymer-lipid hybrid nanoparticles are suitable for said liver targeting: BNP-002 having 5 mM (Molar %) of DLin-MC3-DMA: Cholesterol: PBD-b-PEO (49:39:12); PCL-008 (e.g., PCL-PEO: DMPC: MC3: CHOL (10:10: 45.0:35.0) and/or PCL-012 (e.g., PCL-PEO: DMPC: MC3: CHOL (8:4: 49.0:39.0);
      c) the following polymer-lipid hybrid nanoparticles are suitable for said spleen targeting: BNP-002.2 having 5 mM (Molar %) of DLin-MC3-DMA: Cholesterol: DSPC: PBD-b-PEO (49.3:39.0:10.1:1.6).
21. The polymer-lipid hybrid nanoparticle according to any one of preceding items, wherein said polymer-lipid hybrid nanoparticle is selected from the group consisting of:
   a) BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2);
   b) BNP-002 having 5 mM (Molar %) of DLin-MC3-DMA: Cholesterol: PBD-b-PEO (49:39:12);
   c) BNP-002.2 having 5 mM (Molar %) of DLin-MC3-DMA: Cholesterol: DSPC: PBD-b-PEO (49.3:39.0:10.1:1.6);
   d) PCL-008 having PCL-PEO: DMPC: MC3: CHOL (10:10: 45.0:35.0);
   e) PCL-012 having PCL-PEO: DMPC: MC3: CHOL (8:4:49.0:39.0).
22. The polymer-lipid hybrid nanoparticle according to any one of preceding items, wherein said polymer-lipid hybrid nanoparticle having an organ tropism (e.g., preference for a particular organ, e.g., preference for delivering and/or interacting to/with a particular organ), preferably said organ tropism is selected from the group consisting of: liver tropism, spleen tropism and lung tropism.
23. The polymer-lipid hybrid nanoparticle according to any one of preceding items, wherein said polymer-lipid hybrid nanoparticle consisting of less than five components, preferably consisting of three or four components, further preferably wherein said polymer-lipid hybrid nanoparticle comprising a block copolymer according to any one of preceding items (e.g., PBD-b-PEO or PCL-PEO).
24. The polymer-lipid hybrid nanoparticle according to any one of preceding items, wherein said polymer-lipid hybrid nanoparticle consisting of five or more components, wherein said polymer-lipid hybrid nanoparticle comprising a block copolymer according to any one of preceding items (e.g., PBD-b-PEO or PCL-PEO).
25. A composition comprising a polymer-lipid hybrid nanoparticle according to any one of preceding items, preferably said composition comprising one or more polymer-lipid hybrid nanoparticles according to any one of preceding items.
26. The composition according to any one of preceding items, wherein said composition is a pharmaceutical or diagnostic composition.
27. The composition according to any one of preceding items, wherein said composition is a therapeutic, immunogenic, antigenic or immunotherapeutic composition.
28. The composition or polymer-lipid hybrid nanoparticle according to any one of preceding items comprising one or more oligonucleotide/s, nuclease/s and guide RNA/s for modifying and/or engineering and/or interfering with a genetic material (e.g., genome and/or transcriptome) or template/s (e.g., nucleotide sequence, e.g., RNA or DNA) inside a cell.
29. The composition according to any one of preceding items, wherein said composition is a non-viral delivery system capable of delivering nucleotides to inside a cell.
30. The composition according to any one of preceding items, wherein said composition is a vaccine.
31. Isolated antigen presenting cells or a hybridoma cell exposed to the polymer-lipid hybrid nanoparticle or composition according to any one of preceding items.
32. The antigen presenting cells according to any one of preceding items, wherein said antigen presenting cells comprise a dendritic cell.
33. The antigen presenting cells according to any one of preceding items, wherein said antigen presenting cells comprise macrophages.
34. The antigen presenting cells according to any one of preceding items, wherein said antigen presenting cells comprise B-cells.
35. The composition according to any one of preceding items comprising the polymer-lipid hybrid nanoparticle, composition, antigen presenting cells and/or hybridoma according to any one of preceding items, and further comprising a pharmaceutically accepted excipient or carrier.
36. A kit comprising the polymer-lipid hybrid nanoparticle, composition, antigen presenting cells, hybridoma and/or vaccine according to any one of preceding items.
37. A method of eliciting an immune response in a subject (e.g. human), comprising:
   i) providing the polymer-lipid hybrid nanoparticle, composition, antigen presenting cells, hybridoma and/or vaccine according to any one of preceding items to said subject,
   ii) administering said polymer-lipid hybrid nanoparticle, composition, antigen presenting cells, hybridoma and/or vaccine to said subject, preferably said administering is intradermal, intraperitoneal, intramuscular, subcutaneous, intravenous injection, or non-invasive administration to a mucosal surface.
38. A method of delivering nucleotide/s to inside a cell without using viral vector/s as delivery means, said method comprising:
   i) providing the polymer-lipid hybrid nanoparticle or composition according to any one of preceding items;
   ii) contacting said polymer-lipid hybrid nanoparticle or composition with a cell.
39. A method of modifying and/or engineering and/or interfering with a genetic material (e.g., genome or transcriptome) or template/s (e.g., nucleotide sequence, e.g., RNA or DNA) inside a cell, comprising:
   i) providing the polymer-lipid hybrid nanoparticle or composition according to any one of preceding items (e.g. comprising one or more oligonucleotide/s, nuclease/s and guide RNA/s);
   ii) contacting said polymer-lipid hybrid nanoparticle or composition with a cell.
40. The polymer-lipid hybrid nanoparticle, composition, antigen presenting cells, hybridoma, kit and/or vaccine according to any one of preceding items, for use as a medicament and/or in therapy (e.g., veterinary use).
41. The polymer-lipid hybrid nanoparticle, composition, antigen presenting cells, hybridoma, kit and/or vaccine according to any one of preceding items, for use in one or more of the following methods:
   i) in a method of treating and/or preventing a disease or disorder;
   ii) in a method of antibody discovery and/or screening and/or preparation;
   iii) in a method of production or preparation of an immunogenic or immunostimulant composition;
   iv) in a method of targeted delivery of one or more polypeptides encoded by said polynucleotide, further most preferably said targeted delivery is carried out in a subject;
   v) in a method of stimulating an immune response against said one or more polypeptides encoded by said polynucleotide;
   vi) in a method of triggering cross-protection induced by CD8 (+) T cell-mediated immune response;
   vii) in a method of triggering an immune response comprising a CD8 (+) T cell-mediated immune response and/or CD4 (+) T cell-mediated immune response;
   viii) in a method for treatment, amelioration, prophylaxis and/or diagnostics of an infectious disease, preferably said infectious disease is a viral or bacterial infectious disease; further preferably said viral infectious disease is selected from a group consisting of: influenza infection, PED virus infection, food and mouth virus infection, respiratory syncytial virus infection, herpes virus infection;
   ix) in a method for treatment, amelioration, prophylaxis or diagnostics of a cancer or an autoimmune disease;
   x) in a method for sensitizing cancer cells to chemotherapy;
   xi) in a method for induction of apoptosis in cancer cells;
   xii) in a method for stimulating an immune response in a subject;
   xiii) in a method for immunizing a non-human animal;
   xiv) in a method for preparation of hybridoma;
   xv) in a method for modifying and/or engineering and/or interfering with a genetic material (e.g., genome and/or transcriptome) or template/s (e.g., nucleotide sequence, e.g., RNA or DNA) inside a cell;
   xvi) in a method for delivering nucleotide/s to inside a cell without using viral vector/s as delivery means;
   xvii) in a method for targeting antigen presenting cell/s (e.g., said method not comprising attaching ligand/s to said polymer-lipid hybrid nanoparticle);
   xviii) in a method for targeting cell/s, preferably said cell/s are not antigen presenting cell/s (e.g., said method comprising attaching ligand/s to said polymer-lipid hybrid nanoparticle);
   xix) in a method for maintaining long-term stability and/or potency of said polynucleotide (e.g., siRNA, an mRNA (e.g., SEQ ID NOs: 1, 2 or 3), guide RNA or self-amplifying mRNA (saRNA));
   xx) in a method according to any one of preceding i)-xvix), wherein said method is in vivo and/or ex vivo and/or in vitro method.
42. Use of the polymer-lipid hybrid nanoparticle, composition, antigen presenting cells, hybridoma, kit and/or vaccine according to any one of preceding items for/in one or more of the following:
   i) treating and/or preventing a disease or disorder;
   ii) antibody discovery and/or screening and/or preparation;
   iii) production or preparation of an immunogenic or immunostimulant composition;
   iv) targeted delivery of one or more polypeptides encoded by said polynucleotide, further most preferably said targeted delivery is carried out in a subject;
   v) stimulating an immune response against said one or more polypeptides encoded by said polynucleotide;
   vi) triggering cross-protection induced by CD8 (+) T cell-mediated immune response;
   vii) triggering an immune response comprising a CD8 (+) T cell-mediated immune response and/or CD4 (+) T cell-mediated immune response;
   viii) treatment, amelioration, prophylaxis and/or diagnostics of an infectious disease, preferably said infectious disease is a viral or bacterial infectious disease; further preferably said viral infectious disease is selected from a group consisting of: influenza infection, PED virus infection, food and mouth virus infection, respiratory syncytial virus infection, herpes virus infection;
   ix) treatment, amelioration, prophylaxis or diagnostics of a cancer or an autoimmune disease;
   x) sensitizing cancer cells to chemotherapy;
   xi) induction of apoptosis in cancer cells;
   xii) stimulating an immune response in a subject;
   xiii) immunizing a non-human animal;
   xiv) preparation of hybridoma;
   xv) modifying and/or engineering and/or interfering with a genetic material (e.g., genome and/or transcriptome) or template/s (e.g., nucleotide sequence, e.g., RNA or DNA) inside a cell;
   xvi) delivering nucleotide/s to inside a cell without using viral vector/s as delivery means;
   xvii) targeting antigen presenting cell/s (e.g., said use not comprising attaching ligand/s to said polymer-lipid hybrid nanoparticle);
   xviii) targeting cell/s, preferably said cell/s are not antigen presenting cell/s (e.g., said use comprising attaching ligand/s to said polymer-lipid hybrid nanoparticle);
   xix) for maintaining long-term stability and/or potency of said polynucleotide (e.g., SiRNA, an mRNA (e.g., SEQ ID NOs: 1, 2 or 3), guide RNA or self-amplifying mRNA (saRNA));
   xx) said use according to any one of preceding i)-xix), wherein said use is in vivo and/or ex vivo and/or in vitro use.
43. The method or use according to any one of preceding items, wherein said method or use is in vivo and/or ex vivo and/or in vitro method or use, preferably wherein said method or use is an organ-specific method or use, further preferably said method or use is predominantly (e.g., at least 51%, e.g., at least 55%, at least 60%, at least 65%, least 70%, least 75%, least 80%, least 85%, least 90%, least 95%, least 90%, least 95%, least 98% or least 99%) suitable for targeting or is carried out in liver, spleen and/or lungs (e.g., cell/s and/or tissue/s).

### EXAMPLES OF THE INVENTION

In order that the invention may be readily understood and put into practical effect, some aspects of the invention are described by way of the following illustrative non-limiting examples.

### Example 1: Polymer-Lipid Hybrid Nanoparticles Comprising a Lipid and a Block Copolymer, Methods of Making them, Characterisation and Uses Thereof

### Materials and Methods

### Materials

All solvents and chemicals were purchased from Merck and used as received unless otherwise mentioned. Block copolymers including PBD_{1.2k}-b-PEO_{0.6k} (PBD-PEO) and PCL_{3.3k}-b-PEO _{1K}(PCL-PEO), PLGA (LA: GA=85:15) 1.9K-PEO1K were synthesized in the lab (e.g., Table 1). 1,2-Dimyristoyl-sn-glycero-3-phosphocholine (DMPC, Avanti), 1,2-Distearoyl-sn-glycero-3-phosphocholine (DSPC, Avanti), 1,2-dioleoyl-sn-glycero-3-phosphoethanolamine (DOPE, Avanti), DLin-MC3-DMA (MC3, Avanti), DMG-PEG-2K (DMG-PEG) and Cholesterol (Chol) were bought from Merck. 1, 1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200) was purchased from Corden pharm. EZ Cap™ Firefly Luciferase mRNA (Luc mRNA-SEQ ID NO: 1) was purchased from APExBIO. CD19 mRNA (SEQ ID NO: 3) and OVA mRNA (SEQ ID NO: 2) were bought from Trilink Biotech and stored at −80° C. Quant-iTRiboGreen RNA assay, Lipofectamine™ MessengerMAX™, MultiTox-Fluor™ Multiplex Cytotoxicity Assay, ONE-Glo™ Luciferase Assay were bought from Thermofisher. Human embryonic kidney (HEK293T) cell lines (CRL-11268™) were obtained from ATCC, U.S.A., and cultured according to ATCC's recommendation. The cells were cultured in Roswell Park Memorial Institute (RPMI 1640) medium enriched with 10% fetal calf serum, 100 U/mL penicillin and 100 μg/mL streptomycin (HyClone, U.S.A.) at 37° C. with 5% CO2.

### Methods

Fabrication of mRNA Loaded Polymer Lipid Hybrid Nanoparticles

### Solvent Dispersion Method

Polymer lipid hybrid nanoparticles encapsulation mRNA was prepared by the solvent dispersion method, which was followed by dialysis. Briefly, polymers, lipid, ionized lipid and cholesterol were dissolved in ethanol at predetermined molar ratios with a total concentration of 5 mM (Table 2).

TABLE 2
Physiochemical characterization of Luc mRNA encapsulated by PBD-PEO lipid hybrid
nanoparticles (BNPs) prepared by the solvent dispersion method. Formulations
No. 2 and 8 have been investigated for in-vitro and in-vivo transfection and
encapsulation efficiency; PDI: polydispersity; EE: encapsulation efficiency;

| | | | | | Zeta | | |
|---|---|---|---|---|---|---|---|
| | | | Size | | potential | Loading | |
| No. | S.N. | Composition | (nm) | PDI | (mV) | (μg/ml) | % EE |
| 1 | LNP- | DMG- | 143.6 | 0.17 | 11.1 | 75.5 | 37.7 |
| | OnP | PEG:DSPC:MC3:CHOL | | | | | |
| | | (1.6:10.1:49.3:39.0), 5 mM | | | | | |
| 2 | BNP- | PBD-PEO:MC3:CHOL | 130-150 | 0.15-0.20 | 25-35 | 20-30 | 65-85 |
| | 002 | (12:49:39), 5 mM | | | | | |
| 3 | BNP- | PBD-PEO:DOPE:C12- | 120-140 | 0.10-0.19 | 25-40 | 15-55 | 10-50 |
| | 008 | 200:CHOL | | | | | |
| | | (12:6.5:35:46.5), 5 mM | | | | | |

The aqueous solution was prepared in 20 mM acetic acid buffer (pH 5.0) with mRNA (Luc mRNA or OVA mRNA or CD19 mRNA). Ethanol phase was slowly injected into aqueous phase at a 3:1 ratio with vortex using a vortex mixer. The nanoparticles formed while vortexing and then dialysed against buffer (20 mM Trisbuffer, 4.5 mM Acetate, 5% Sucrose, pH 7.4) overnight at 4° C. overnight using dialysis membrane (300 kDa molecular weight cut-off (MWCO) cellulose ester membrane, Spectrum Laboratories Inc., cat. no. 131450) to remove organic solvents and unencapsulated mRNA. The dialyzed solution was sterile filtered using 0.22 μm sterile filter (Sartorius) and stored at 4° C. Lipid nanoparticles (LNP) encapsulating mRNA was prepared using a similar molar composition as reported in the literature and used as control, where the molar ratio among ionized lipid/cholesterol/DSPC/DMG-PEG-2Kis set 49:39: 10.5:1. 5. The N/P ratio ((N in the ionized cationic lipid and P in mRNA) was ranged from 4-40 in this method.

### Precision NanoSystems Incorporation (PNI) NanoAssembl Platform (can Also be Referred to as the PNI Method)

Polymer Lipid Hybrid Nanoparticles Encapsulation mRNA Produced by PNI

Polymer lipid hybrid nanoparticles encapsulation mRNA was produced in a similar molar composition as presented above by Precision Nanosystem Incorporation Nanoasemblr system (Ignite™, PNI, Canada)). Briefly, ethanol phase containing mixture of polymers and lipids with predetermined molar ratios (see e.g., Tables 2 and 3) and mRNA containing aqueous phase (20 mM acetic acid buffer, pH 5.0) injected simultaneously in a Y-shaped staggered herringbone micromixer of 300 μm width and 130 μm height). Nanoparticles were produced at 5 mM polymer/lipid concentration, 3:1 aqueous: organic flow rate ratio (FRR), 12 mL/min total flow rate (TFR). The nanoparticles were dialyzed against buffer (20 mM TRIS, 4.5 mM Acetate, 5% sucrose, pH 7.4) overnight at 4° C. using 300 kDa molecular weight cut-off (MWCO) cellulose ester membrane, Spectrum Laboratories Inc., cat. no. 131450) with magnetic stirring. The dialyzed solution was sterile filtered using 0.22 μm sterile filter (Sartorius) and stored at 4° C. for further usage.

### Polymer Lipid Hybrid Nanoparticles Encapsulation Cas12a/gRNA Produced by the PNI System

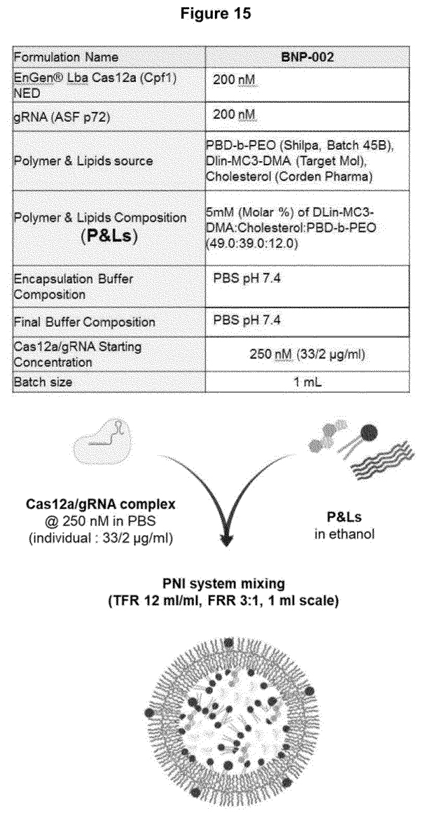

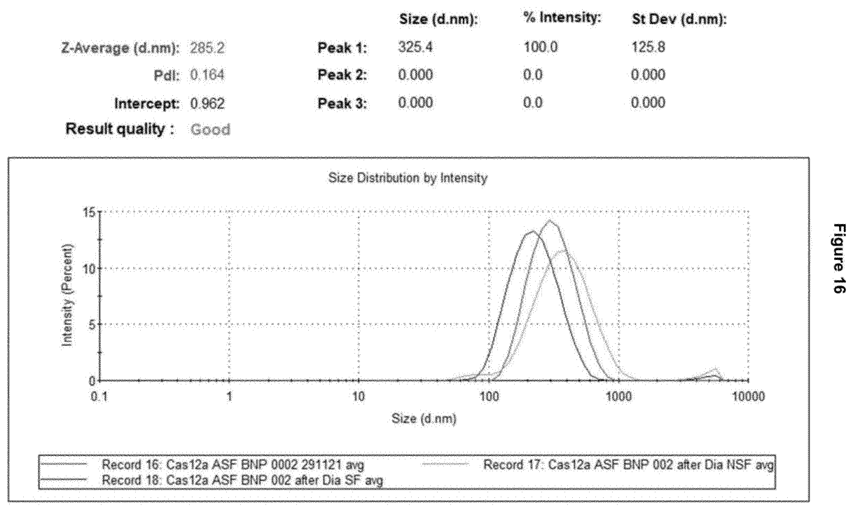

EnGen@ Lba Cas12a (Cpf1) NED (Cas12a) and gRNA with ASF p52 were mixed at 250 nM concentration. The solution was incubated at room temperature (RT) for 10-15 mins for Cas12a to bind to gRNA. This was further encapsulated in the BNP-002 (Table 2) using the PNI system with TFF 12 ml/min and FRR of 3:1 at 1 ml scale (FIG. 15). After the formulations were complete, DLS of the samples was done and rest of the samples was put on dialysis with PBS buffer. After dialysis sample was harvested and DLS was collected before and after sterile filtration.

### Dynamic Light Scattering Analysis

The particle size, polydispersity and zeta potential of the nanoparticles was measured by ZetasizerNano ZS system (Malvern Instrument Ltd., Malvern, UK) equipped with a He-Ne laser beam at 658 nm (scattering angle: 90°). A 50 μL of sample was diluted 10 times using dialyzed buffer and an average of 3 measurements (10 runs per measurement) was collected and the data were presented as average.

### Cryogenic-Transmission Electron Microscopy (Cryo-TEM)

4 μL of the mRNA encapsulated nanoparticles (5 mM) was adsorbed onto a lacey holey carbon-coated Cu grid, 200 mesh size (Electron Microscopy Sciences). The grid was surface treated for 20 s via glow discharge before use. After surface treatment, a 4 μL sample was added and the grid was blotted with Whatman filter paper (GE Healthcare Bio-Sciences) for 2 s with blot force 1 and then plunged into liquid ethane at −178° C. using a Vitrobot (FEI Company). The cryo-grids were imaged using a FEG 200 keV transmission electron microscope (Arctica; FEI Company) equipped with a direct electron detector (Falcon III; Fei Company).

Quantification of mRNA Encapsulated in the Nanoparticles

mRNA encapsulated in the nanoparticles was quantified using a modified Quant-iTRiboGreen RNA assay. A 20 μL portion of mRNA encapsulated nanoparticles or free mRNA at known concentrations was added to a 384-well black plate. A 10 μL amount of TE buffer or TE buffer with 10% Triton-X 100 was added into each well, and the plate was incubated for 15 min at 37° C. to lyse ACM vesicles before adding 20 μL of 1×Quant-iT RiboGreen (Invitrogen, Thermo Fisher Scientific). Each sample and standard were prepared in triplicate. The plate was incubated for 10 min at 25° C., and fluorescence was measured (excitation, 500 nm; emission, 525 nm) using a plate reader (Biotek). The concentration of mRNA was calculated according to the standard curve. Encapsulation efficiency (EE) was calculated as (FI-Fo)/F*100, where Ft and Fo are the amount of mRNA quantified in presence and absence of 1% triton X-100, F; was the initial amount of mRNA used for preparing nanoparticles.

### Gel Electrophoresis Assay

Gel electrophoresis was used to analyse the integrity of mRNA in various formulations. Agarose was dissolved in 1 xTris-acetate-EDTA (TAE) buffer to form a 1% w/v solution upon heating, followed by the addition of SybrSafe Dye (5 μl per 50 mL). The solution was mixed well and pour on to the casting tray with comb placed on it. The gel was allowed to solidify. The comb was removed, and the gel was placed on buffer tank with 0.5× TAE buffer. 20 UL of mRNA encapsulated nanoparticles (mRNA equal amount 500 ng) were mixed with 2 μL of 20% Triton X for 30 min, followed by the addition of 20 μl of 2× RNA loading dye. The samples were heated at 70° C. for 10 mins on heat block. The samples were cool down and then loaded on the gel (20 μ per well). It is noted that RNA Ladder was used as molecular weight standard. A power supply was connected to the chamber and a voltage of 80 V applied for 40-45 mins. The gel was then visualized using an ImageQuant LAS 500 system.

### RNase Protection Assay

Tested samples (20 μL) were challenged with RNase (1 μl of 10 μg/ml stock), followed by incubation at 37 degrees for 20 mins. Subsequently, 0.2% SDS (4 μl of 1% SDS stock) was added to each sample. It is noted that 0.2% SDS works as RNAse inhibitor. Positive control samples were incubated with 1% of triton X at room temperature for 30 min. For negative control groups, samples (20 μL) were challenged with RNase (1 μl of 10 μg/ml stock), followed by incubation at 37° C. for 20 mins. Subsequently, 2% Triton X (2 μl of 20% Triton X) was added to each sample. The mixture was incubated at room temperature for 30 min. addition of 20 μl of 2× RNA loading dye. The samples were heated at 70° C. for 10 mins on heat block. The samples were cool down and then loaded on the gel (20 μl per well). It is noted that RNA Ladder was used as molecular weight standard. A power supply was connected to the chamber and a voltage of 80 V applied for 40-45 mins. The gel was then visualized using an ImageQuant LAS 500 system.

mRNA Transfection

HEK293T cells (CRL-11268™) were seeded in 96 well plates at a density of 25000 cells per well. After overnight incubation at 37° C., the cells were transfected with Luc mRNA encapsulated nanoparticles or control Luc mRNA using Lipofectamine™ MessengerMAX™ (MM, Thermo Fisher). The cytotoxicity of free mRNA and mRNA-loaded nanoparticles against HEK293 cells was determined after 24-h incubation through MultiTox-Fluor™ Multiplex Cytotoxicity Assay. Luciferase activity was measured with the ONE-Glo™ Luciferase Assay according to the manufacturer's instructions (Promega).

HEK293T cells were seeded in 24 well plates at 250,000 cells per well. After overnight incubation, the cells were transfected with OVA mRNA nanoparticles (OVA mRNA equal amount: 1 μg) or control OVA mRNA (100 ng and 200 ng) using Lipofectamine™ Messenger MAX™ (Thermo Fisher). The cells were collected after 24 h transfection. The cells were lysed, and protein was quantified using BCA assay (Thermo Fisher) according to manufacturer's protocol. 50 μL of sample (containing 150 ng of protein) was mixed with 50 μL of loading buffer and the mixture was heated at 95° C. for 10 min. The samples (20 μL) were loaded for SDS-PAGE. The OVA protein was then detected by western blotting with a monoclonal antibody against the OVA protein. The gel was then visualized using an ImageQuant LAS 500 system.

In Vivo Luc mRNA Delivery

The animal studies were approved by Institutional Animal Care and Use Committee, A-star, Singapore. 6-8-week-old female C57BL/6 mice were randomly grouped. C57BL/6 mice were injected with Luc mRNA with dose of 0.35 mg/kg by IM (thigh muscle), SC (flank) and IV (tail vein), respectively. There were 6 groups for each administration route3 mice per group, total 18 mice per administration route). In the case of intramuscularly (IM) administration route, mice were intramuscularly (IM) injected at the inner thigh with Luc mRNA encapsulated within LNP-ON, BNP-002, BNP-008, PCL-008 and PCL-012 at a dosage of 0.35 mg/kg, where PBS was used as negative control and LNP-ON was used as positive control for comparison. At 6 h post-injection, mice were anesthetized with 2% isofluorane in oxygen and imaged 10 min after intraperitoneal injection of D-Luciferin (150 mg/Kg). Bioluminescence imaging was performed using an IVIS Spectrum imaging system. Organs collected for ex vivo imaging. Mice were imaged at 10 minutes post administration of D-luciferin. Bioluminescence values were quantified by measuring photon flux in the region of interest using the Living IMAGE Software provided by Caliper.

Mice Injected with ACM-OVA mRNA Polymer-Lipid Hybrid Nanoparticles Expressed OVA Peptide on Surfaces of Dendritic Cells

The animal studies were approved by Institutional Animal Care and Use Committee, A-star, Singapore. 6-8-week-old female C57BL/6 mice were intramuscularly (IM) injected at the inner thigh with 3-4 μg OVA mRNA encapsulated within LNP or ACM carrier. Two days after, animals were sacrificed and inguinal lymph nodes that drain the site of injection were harvested. To release DCs for analysis, lymph nodes were cut into tiny pieces and digested with 0.2 mg/ml collagenase and 0.05 mg/ml DNAse I in complete RPMI medium for 30 min at 37° C. Cells were passed through 70 μm cell strainers. To prepare for flow cytometry, cells were stained using the following antibody panel: BUV395-CD45, FITC-CD3, FITC-CD19, FITC-CD49b, BV510-MHC-II, BV650-CD64, PE-CD594-CD11c, PerCP-Cy5.5-XCR1, APC-CD172a, APC-Cy7-CD86, and PE-SIINFEKL-H2kb. Live/dead discrimination was done using fixable viability dye eFluor 455 UV. Non-specific staining was reduced using mouse FcR block reagent. Cells were analysed using LSR Fortessa (BD) and data was analysed using FlowJo V10.

OVA mRNA Vaccine Adaptive Immunity Study

Mouse vaccination. The animal studies were approved by Institutional Animal Care and Use Committee, A-star, Singapore. 6-8 weeks old, female C57BL/6 (n=5 per group) were intramuscularly (IM) injected at each thigh muscle with total of 5 μg OVA mRNA on Days 0 and 14. Blood was collected by retro-orbital puncture on Day 7 and 21 for assessment of circulating T cells and Days 14 and 24 for serum IgG titres.

T cell analysis. Blood was collected in 0.1% EDTA. Cells were pelleted at 500 g, 40 C, 5 min and erythrocytes were lysed using RBD lysis buffer (Thermo Fisher). White blood cells were surface stained with antibodies and pentamer for analysis by flow cytometry (Table 7). Cells were acquired on LSR II cytometer (BD) and data analysed using FlowJo V10 software.

TABLE 7
Antibodies and pentamers used for analysis by flow cytometry.

| | | | mAb | | Catalogue |
|---|---|---|---|---|---|
| # | Target | Colour | clone | Manufacturer | number |
| 1 | CD45 | BUV395 | 30-F11 | BD | 564279 |
| 2 | CD3 | BV785 | 17A2 | BioLegend | 100232 |
| 3 | CD4 | AF700 | GK1.5 | BioLegend | 100430 |
| 4 | CD8 | APC- | 53-6.7 | eBioscience | 47-0081-82 |
| | | eFluor | | | |
| | | 780 | | | |
| 5 | H2Kb- | PE | Pentamer | Proimmune | F093-2B-G |
| | SIINFEKL | | | | |

OVA IgG titre. 96-well Corning EIA/RIA plate was coated with 2 μg/ml OVA protein overnight at 40 C. The next morning, the plate was washed thrice with PBS+0.1% v/v Tween-20 before blocking with 2% w/v BSA in wash buffer for 1.5 h at 370 C. Serum was serially diluted with Assay Diluent (PBS+0.5% w/v BSA+0.1% v/v Tween-20) and applied to corresponding wells of the ELIA plate. Samples were incubated 1 h at 370 C before the plate was washed thrice. HRP-conjugated goat anti-mouse IgG (H+L) (BioRad) was diluted 1:10,000 and applied to the ELISA plate. The plate was incubated 1 min at 370 C before washing thrice. To visualize antibody binding, TMB substrate (Sigma Aldrich) was added and incubated 30 min at room temperature. The reaction was stopped with Stop Solution (Thermo Fisher) and absorbance at 450 nm measured. Data was analyzed using 5-parameter non-linear regression (GraphPad Prism version 9.1.2). Antibody titer, defined as the reciprocal of the highest dilution giving an OD value 3X background, was interpolated from the titration curve.

### Statistical Analyses

Statistical significance was evaluated via an independent two-tailed Student's t test or two-way ANOVA. P-values less than 0.05 were considered statistically significant. Data were analyzed using GraphPad Prism 7 software.

### Results and Discussion

Physicochemical Characterization of mRNA Loaded Polymer-Lipid Hybrid Nanoparticles Prepared by Solvent Dispersion Method (Co-Solvent Method/Nano-Preciptation Method)

BNPs composed of PBD-PEO, MC3 and Chol encapsulating mRNA were first prepared by solvent dispersion method. LNP-onpattro (LNP-ON or LNP-ONP) containing DMG-PEG, DSPC, MC3 and Chol (1.6:10.1:49.3:39.0) with mRNA was prepared via the same method and used as control. The physicochemical properties of nanoparticles including particle size, polydispersity, zeta potential, mRNA encapsulation efficiency, loading concentration were summarized in Table 2. BNP-002 has an average particle size of 138 nm with a relative lower polydispersity (PDI: 0.176). It is noted that the hydrodynamic diameter of LNP-ON was 158 nm and the PDI of LNP-ON was 0.17. The results indicated that the particle size and PDI of BNP-002 are comparable to those of LNP-ON. The surface potential of BNP-002 was 27.8 mV, which is significanlty higher than that of LNP-ON (11.1 mV). This was in line with the fact that the N/P value of BNP-002 was significanlty higher than that of LNP-ON (27). To explore the potential of mRNA loaded nanoparticles with tumor antigens for cancer immunotherapy, OVA mRNA was loaded into BNPs via solvent dispersion method. Interestingly, it was found that OVA mRNA BNPs showed an diameter value of 107 nm with a low PDI value of 0.137. The morphology of Luc mRNA loaded BNPs and OVA mRNA loaded BNPs and were analyzed by cryo-TEM, as illustrated in FIG. 1, Luc mRNA loaded BNPs formed spherical particles and exhibited stacked bilayer structure. It is interesting to note that OVA mRNA loaded BNPs displayed similar stacked bilayer structure (FIG. 2). At N/P value of 27, all mRNA moleucles are complexted with the positively charged ionizable lipid (MC3), which may led to the formation of stacked bilayer structure. The amphiphilicbilayer forming polymer PBD-PEO is hypothesized to provide the outer layer of the mRNA BNPs to stabilize the internal mRNA-ionizable lipid stacked bilayer structure. The structure of mRNA BNPs is in agreement with LNPs containing mRNA,where LNP-mRNA composed of f KC2, DSPC, Chol, PEG-lipid at a molar composition of 50/10/38.5/1.5 exhibited superficial bilayer and stacked bilayer internal structure. The mRNA encapsulation efficiency and loading concentration were evaluated by Ribogreen assay. The results indicated that physiochemical properties of BNPs were not significantly affected by different types of mRNA. Encapsulation efficiency was evaluated using Ribogreen assay. As listed in Table 2, BNP-002 showed significantly higher encapsulation efficiency (67.8%) as compared to that of LNP-ON (37.7%), whereas its loading concentration was markedly lower than that of LNP-ON (20.3 μg/mL vs 75.5 μg/mL). The lower loading concentration was attributed to incomplete mixing with solvent dispersion method.

The ionized lipid has been reported to play a critical role on the performance of mRNA nanoparticles. Numerous studies have shown that ionized lipids including DLin-KC2-DMA and DLin-MC3-DMA achieved maximum activity on mRNA delivery. It was recently reported by Kauffmann et al. that lipid like material (lipidoid) C12-200 nanoparticles incorporated with DOPE, DMG-PEG and Chol (35:16: 2.5:46.5) (LLNPs) remarkably increased EPO expression seven-fold in serum as compared to the benchmark formulation LNP-ON. Inspired by Kauffmann's design, we integrated PBD-PEO with LLNPs and investigated the functionality of resulting Luc mRNA BNPs. BNP based on C12-200 were prepared with solvent dispersion method as listed in Table 2, BNP-008 had an average particle size ranging from 121 to 200 nm, lower PDI values less than 0.22, surface charge ranging from 25 to 30 mV. BNP-008 had the smallest particle size and highest in vitro transfection efficiency (data not shown). It was therefore selected for further studies.

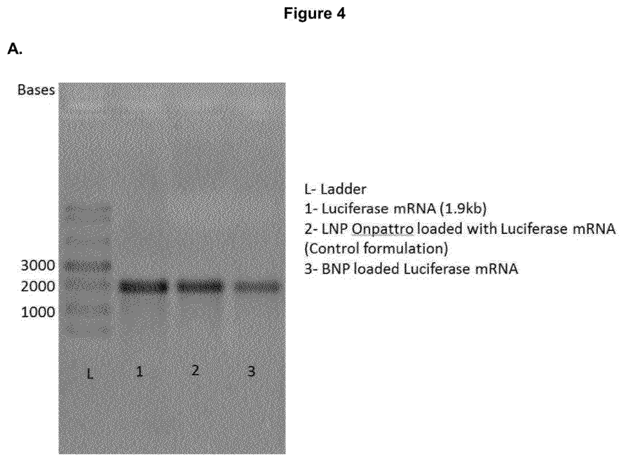

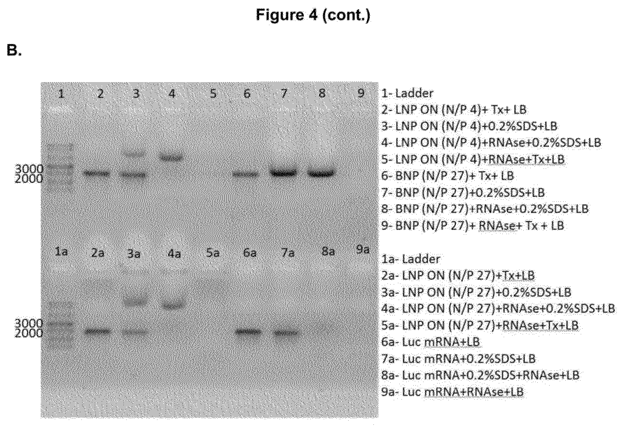

mRNA integrity in BNPs were analyzed by gel electrophoresis. As illustrated in FIG. 4A, Luciferase mRNA remains intact after encapsulated into both BNPs and LNP-ON. It is known that LNPs not only facilitate cellular uptake and expression but also protect mRNA from degradation by exonucleases and endonucleases (RNase). The ability of BNPs to resist the degradation by RNase was assessed by RNase protection assay using gel electrophoresis. As shown in FIG. 4B, mRNA remained intact for both BNPs (lane 6) and LNP-ON (lane 2) after 2 weeks storage at 4° C. However, mRNA was completely degraded upon exposure to RNase in the presence of triton (lane 5 & 9). It is noteworthy that mRNA encapsulated BNPs was protected from degradation in the presence of 0.2% SDS (RNase inhibitor) (Lane 8), whereas mRNA encapsulated LNP-ON was completely degraded in the presence of RNase inhibitor (Lane 4). The results strongly indicated that BNPs were able to protect mRNA from RNase degradation.

It should be mentioned that the BNPs produced by solvent dispersion method had lower encapsulation efficiency and lower loading concentration. To improve the encapsulation efficiency and loading levels of BNPs, mRNA loaded nanoparticles were further prepared by Precision Nanosystem Incorporation (PNI Nanoasemblr Patform.

Physicochemical Properties of mRNA Loaded Nanoparticles Prepared by Precision NanoSystem Incorporation (PNI) Nanoasemblr Patform
Physicochemical Properties of mRNA Loaded Nanoparticles Prepared by the PNI Method

BNPs and PCLs were formulated as specified compositions (e.g., Table 2, Table 3, Table 4 and Table 5) at N/P molar ratio of 10 using the PNI method.

TABLE 3
Physiochemical characterization of Luc mRNA loaded PCL-PEO lipid hybrid nanoparticles
prepared by Precision Nanosystem Incorporation (PNI) Nanoasemblr Patform.

| | | | | | Zeta | | |
|---|---|---|---|---|---|---|---|
| | | | Size | | potential | Loading | |
| No. | S.N. | Composition | (nm) | PDI | (mV) | (μg/ml) | % EE |
| 1 | PCL- | PCL- | 70-90 | 0.15-0.20 | 5-15 | 70-80 | 90-100 |
| | 008 | PEO:DMPC:MC3:CHOL | | | | | |
| | | (10:10:45.0:35.0) | | | | | |
| 2 | PCL- | PCL- | 120-140 | 0.20-0.25 | 5-15 | 80-95 | 85-95 |
| | 012 | PEO:DMPC:MC3:CHOL | | | | | |
| | | (8:4:49.0:39.0) | | | | | |

TABLE 4
Exemplary BNPs compositions

| | Molecular | Mole | wt. | | Amount |
|---|---|---|---|---|---|
| Polymer/Lipids | weight (g/mol) | % | % | mM | (mg) |
| DLin-MC3-DM | 642.1 | 49.0 | 45% | 2.45 | 1.57 |
| Cholesterol | 386.7 | 39.0 | 22% | 1.95 | 0.75 |
| PBD_{1.2k}-b-PEO_{0.6k} | 1900.0 | 12.0 | 33% | 0.60 | 1.14 |

TABLE 5
Buffer Compositions for mRNA loaded nanoparticles
Buffer Composition

| | |
|---|---|
| Buffer for mRNA Encapsulation | 20 mM Acetic buffer, pH 5.0 |
| Final buffer for mRNA BNPs | 20 mM Tris buffer, 4.5 mM |
| | Acetate, 5% Sucrose, pH 7.4 |

Briefly, mRNA diluted in acetic acid buffer (20 mM, pH5: 0) was rapidly mixed with polymer and/or lipids in ethanol at 3:1 aqueous: ethanol volume ratio. The aqueous to organic. flow rate ratio (FRR) was set to be 3:1 and the total follow rate (TFR) was set to be 12 mL/min. Interestingly, BNP-002, BNP-008, PCL-008 and PCL-012 with N/P ratio at: 10 showed 80-130 nm in z-average diameter with lower polydispersity (Tables 3 and 6A). It is noteworthy that all formulations exhibited superior encapsulation efficiency (˜100%) (Table 6B), Benchmark LNP-ON with N/P at 4 prepared by PNI yielded particles with 73 nm, low polydispersity (0.11) and strikingly high encapsulation efficiency (˜100%) (Table 6A and 6B), which is consistent with previous studies. Importantly, all formulations demonstrated high load concentrations (more than 75 μg/mL) (Table 6B), which is significantly increased as compared to those produced by solvent dispersion method.

TABLE 6A
Physiochemical characterization of Luc mRNA loaded
nanoparticles prepared by Precision Nanosystem
Incorporation (PNI) Nanoasemblr Patform

| | LNP | | | | |
|---|---|---|---|---|---|
| Test Sample | Onpattro | BNP- | BNP- | PCL- | PCL- |
| Name | (LNP-ON) | 002 | 008 | 008 | 012 |
| N/P | 4 | 10 | 10 | 10 | 10 |
| Particle Size | 73.1 | 131.8 | 118 | 80.1 | 133.4 |
| (nm) | | | | | |
| Polydispersity | 0.11 | 0.231 | 0.029 | 0.19 | 0.241 |
| Index | | | | | |
| Zeta Potential | 12.4 | 22.7 | 22.9 | 7.5 | 4.57 |
| (mV) | | | | | |
| Starting Conc. | 200 | 80 | 290 | 71 | 84 |
| (μg/mL) | | | | | |
| mRNA Loading | 211.6 | 79.7 | 270.3 | 75.9 | 91.2 |
| (μg/mL) | | | | | |
| Agarose Gel | Intact | Intact | Intact | Intact | Intact |
| | band | band | band | band | band |
| Endotoxin | 4.0 | 2.1 | 2.8 | 2.9 | 3.1 |
| (EU/mL) | | | | | |

TABLE 6B
RiboGreen Assay (Luciferase mRNA) Luc mRNA loaded
nanoparticles prepared by Precision Nanosystem
Incorporation (PNI)Nanoasembir Patform.

| | Conc. without Tx | Conc. with Tx | Loading | |
|---|---|---|---|---|
| Sample | (μg/ml) ¹ | (μg/ml) ² | (μg/ml) ³ | % EE |
| LNP Onpattro | 13.6 | 226.3 | 212.7 | 106.4 |
| NSF | | | | |
| LNP Onpattro SF | 5.3 | 217.0 | 211.6 | 105.8 |
| BNP-002 NSF | 6.5 | 94.7 | 88.2 | 110.3 |
| BNP-002 SF | 3.4 | 83.1 | 79.7 | 99.6 |
| BNP-008 NSF | 22.4 | 343.6 | 321.2 | 110.8 |
| BNP-008 SF | 11.7 | 282.0 | 270.3 | 93.2 |
| BNP-PCL-008 | 6.0 | 89.5 | 83.5 | 117.7 |
| NSF | | | | |
| BNP-PCL-008 SF | 6.6 | 82.5 | 75.9 | 106.9 |
| BNP-PCL-012 | 7.1 | 106.5 | 99.4 | 118.3 |
| NSF | | | | |
| BNP-PCL-012 SF | 6.5 | 97.7 | 91.2 | 108.6 |

¹ This is to measure any free unencapsulated mRNA or mRNA that binds to the surface of the vesicles.
² This is to measure the total mRNA present in the sample as Triton was added to break open the vesicle.
³ mRNA loading is calculated by using the concentration with Triton minus the concentration without Triton.
NSF denotes non-sterile filter.
SF denotes sterile filter (e.g., using PES syringe filter 0.2 μm (Millipore)).

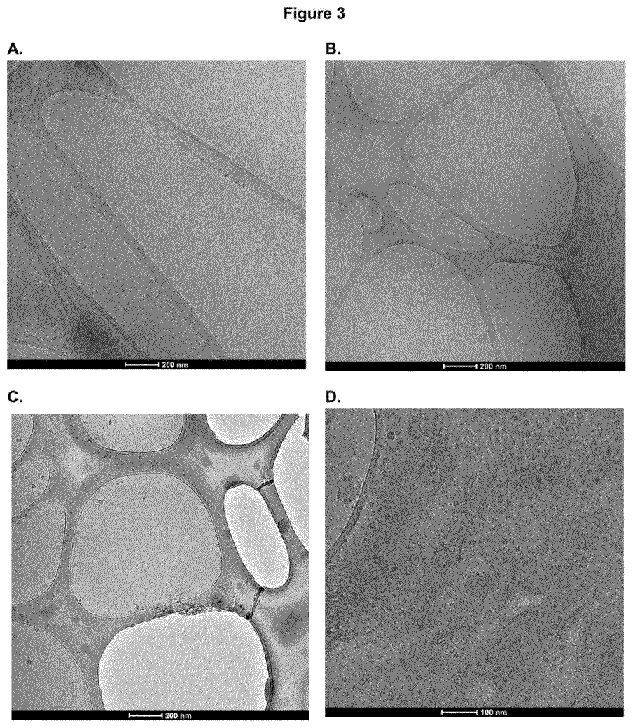

Among these, BNP-008 with: N/P at 10 exhibited strikingly higher loading concentrations, which is comparable to that of benchmark LNP-ON with N/P at 4 (Table 6B). The surface charge of BNP-002 and BNP-008 were ˜ 22 mV, while the surface charge of PCL-008 and PCL-012 were ˜ 5-8 mV (Table 6A). The morphology of BNPs and PCLs were analyzed by cryo-TEM. All formulations formed spherical particles with 50-200 nm in diameter, which agrees with DLS analysis. The nanoparticles had an electro-lucent amorphous internal structure surrounded by a peripheral bilayer (FIG. 3A-D). This structure is consistent with previous reports showing that the mRNA loaded nanoparticle had electron-lucent amorphous core with a peripheral bilayer membrane with high mRNA loadings.

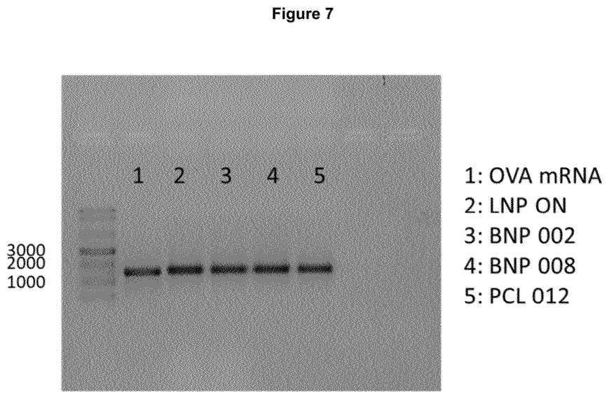

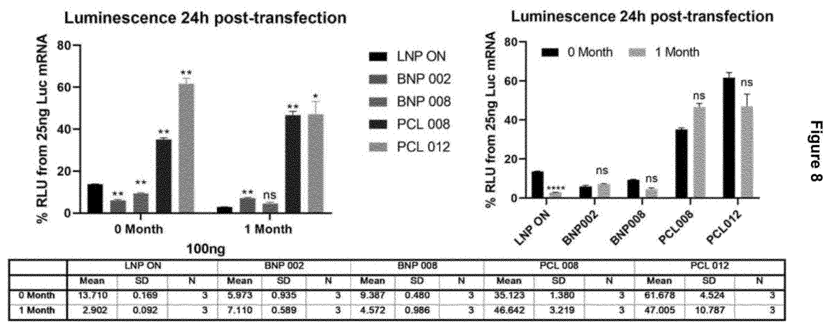

OVA mRNA was also encapsulated into BNPs and PCLs using PNI. All formulations exhibited small particle size, low polydispersity, strikingly higher encapsulation efficiency (˜100%) and high mRNA loading level (data not shown). The integrity of OVA mRNA encapsulated in BNPs and PCLs was assessed by gel electrophoresis. As seen in FIG. 7, OVA mRNA remained intact for all the formulations produced by PNI. We thus concluded that PNI was a more suitable method for BNPs and PCLs manufacturing with superior mRNA encapsulation efficiency (˜100%) and high loading level in this study. Optimal BNPs and PCLs produced by PNIwere selected for in vivo Luc mRNA delivery studies.

### Physicochemical Properties of Cas12a/gRNA Loaded Nanoparticles Prepared by PNI

Cas12a, CRISP effector protein, has shown great promise in the treatment of genetic diseases. CRISPR technology based on Cas12a and gRNA has been reported to be powerful for RNA-based gene regulation. To explore the potential of polymer lipid hybrid nanoparticles as carriers for Cas12a and gRNA, we developed BNP-002 nanoparticles for the encapsulation of Cas12a and gRNA. Cas12a and gRNA with ASF p52 were first mixed and incubate for 10-15 min. This was further encapsulated in the BNP-002 using the PNI system. The resulting nanoparticles were 285.2 nm by dynamic light scattering (FIG. 16) with low PDI (0.164).

In Vitro Transfection Efficiency of mRNA Loaded Nanoparticles Prepared by Solvent Dispersion Method

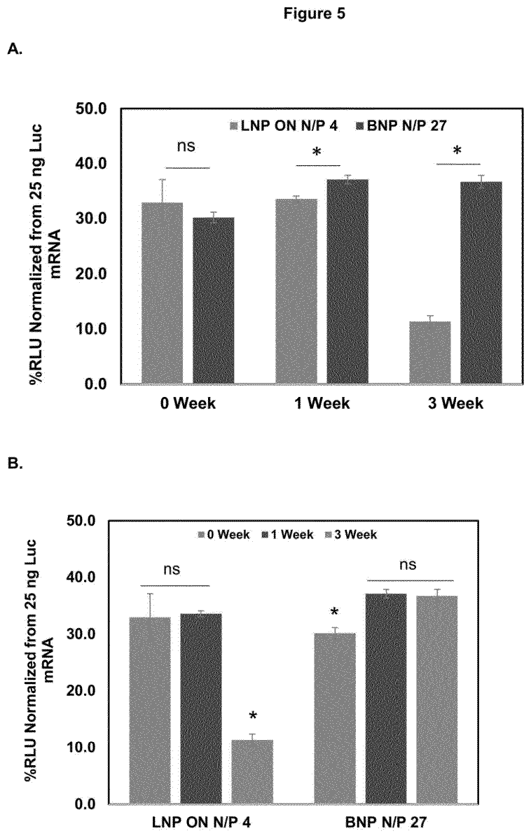

The in vitro delivery efficacy and cytotoxicity of BNPs was investigated by normalized luminescence intensity after transfection of Luc mRNA into HEK293T cells. As shown in FIG. 5, BNP-002 showed high expression of luciferase protein, with up to 30% relative to 25 ng of MM complex in HEK293T cells. Notably, BNP-002 demonstrated comparable in vitro transfection potency as compared to LNP-ON (p>0.1). It is worth noting that BNP-002 has less cytotoxicity as compared to LNP-ON (data not shown). Time dependent luciferase activity of BNPs was further evaluated over 3 weeks course. As revealed by FIG. 5, the BNP-002 has shown significant increased luciferase protein expression after 1 week storage at 4° C. The luciferase activity of BNP-002 was maintained after 3 weeks storage at 4° C. In contrast, in the case of LNP-ON, luciferase protein expression was significantly reduced from 33% at week 1 to 11% at week 3 (p<0.005). The results indicated that BNPs are stable at 4° C. over 3 weeks. The long-term storage stability of BNPs is being monitored.

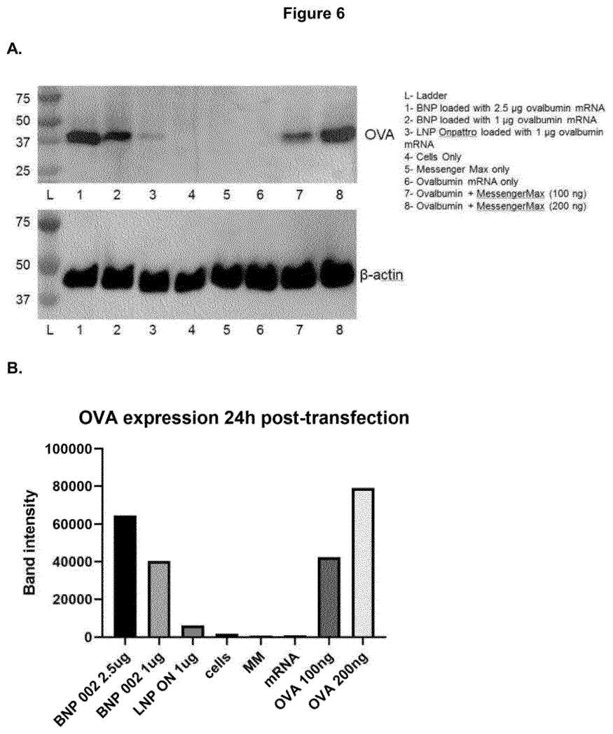

Next, BNPs was validated as nano vaccine to deliver OVA mRNA encoding antigen in HEK293 cells. As illustrated FIG. 6, OVA mRNA loaded BNP-002 yielded dose dependent OVA protein expression. Importantly, OVA mRNA loaded BNP-002 induced significantly higher level of OVA protein expression as compared to that from mRNA formulated with LNP-ON. The results indicated that BNPs outperformed LNP-ON delivering Luc mRNA and OVA mRNA to cells with efficient protein translation.

In Vitro Transfection Efficiency of mRNA Loaded Nanoparticles Prepared by Precision NanoSystem Incorporation (PNI) Nanoasemblr Patform

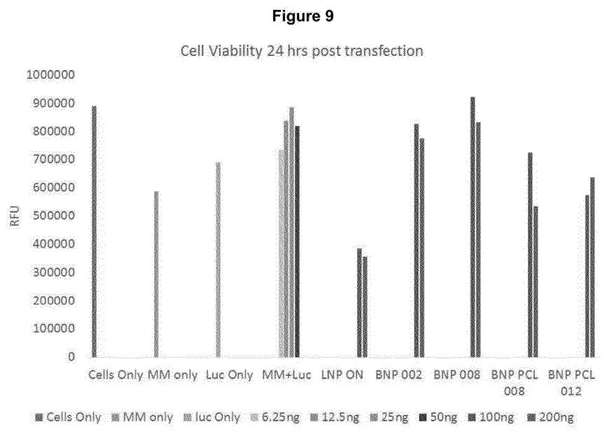

As stated earlier, all formulations produced by PNI demonstrated remarkably higher encapsulation efficiency (˜100%) at N/P ratio of 10. In vitro delivery efficacy of these formulations was evaluated for delivery of Luc mRNA in HEK293T cells (FIG. 8). BNP-002 and BNP-008 induced lower normalized luminance intensity as compared to that of LNP-ON (p<0.05). However, PCL-008 and PCL-012 at N/P ratio of 10 promoted Luc mRNA transfection and the potency significantly increased 2.7-fold and 4.5-fold, respectively, as compared to that of LNP-ON at N/P ratio of 4 (p<0.01). Moreover, ACM formulations (BNPs and PCLs) showed less cytotoxicity as compared to LNP-ON (FIG. 9). Next, long-term stability of the formulations was evaluated over a month of storage at 4° C. As seen in FIG. 8, there is no significant difference in in vitro transfection efficacy of Luc mRNA among BNPs and PCLs over 1 month storage (p>0.05). In contrast, the transfection efficacy of Luc mRNA encapsulated in LNP ON was markedly reduced after 1 month storage (p<0.05). The results indicated BNPs and PCLs produced by PNI exhibited high thermostability at 4° C.

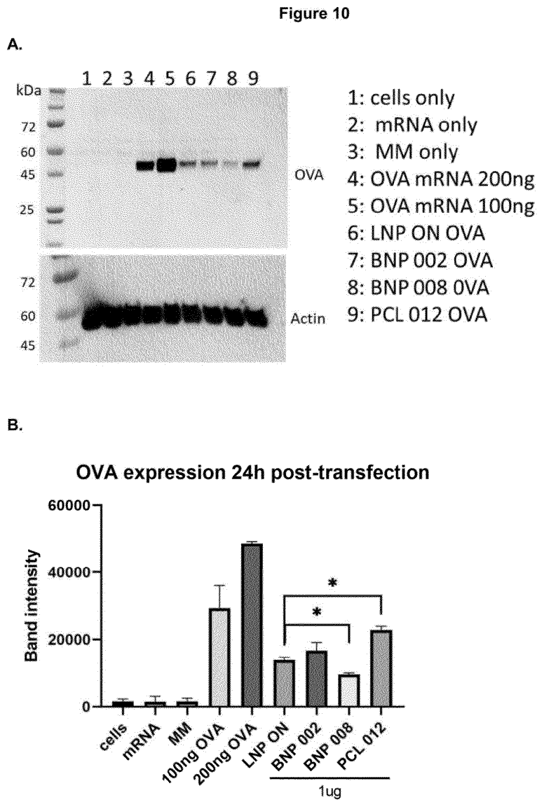

HEK293T cells were treated with OVA mRNA formulations and the OVA protein expression was assessed by western blot assay. As illustrated in FIG. 10, there is no significance difference in OVA protein expression between BNP-002 and LNP-ON, whereas PCL-012 induced significantly higher OVA protein levels as compared to LNP-ON (p<0.05). On the other hand, BNP-008 yielded lower OVA protein than that of LNP-ON (p<0.05).

In Vivo Delivery Efficacy of Luc mRNA

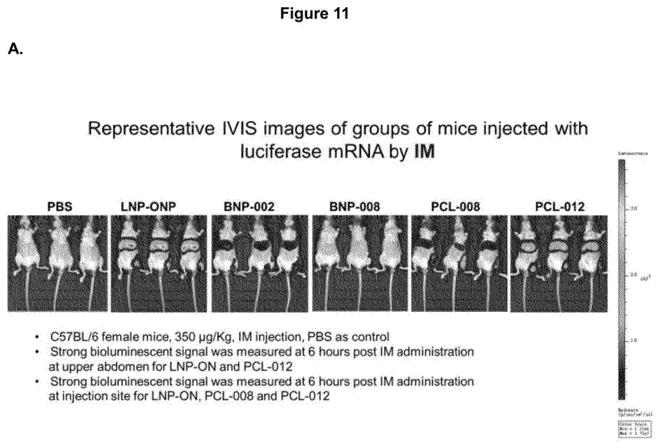

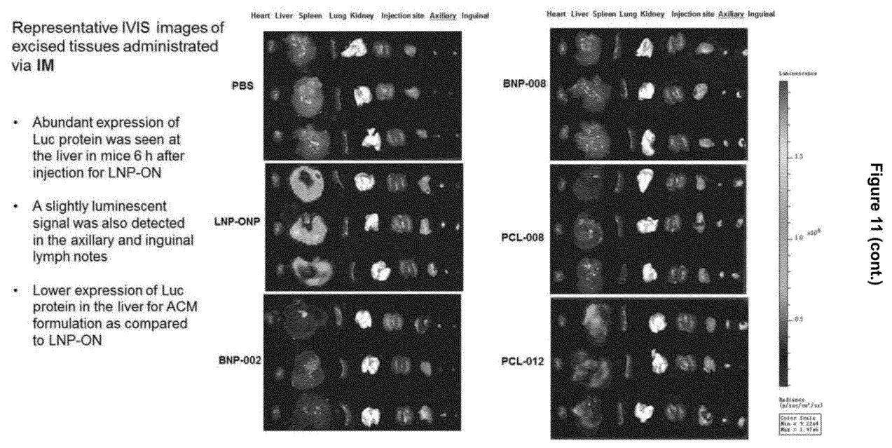

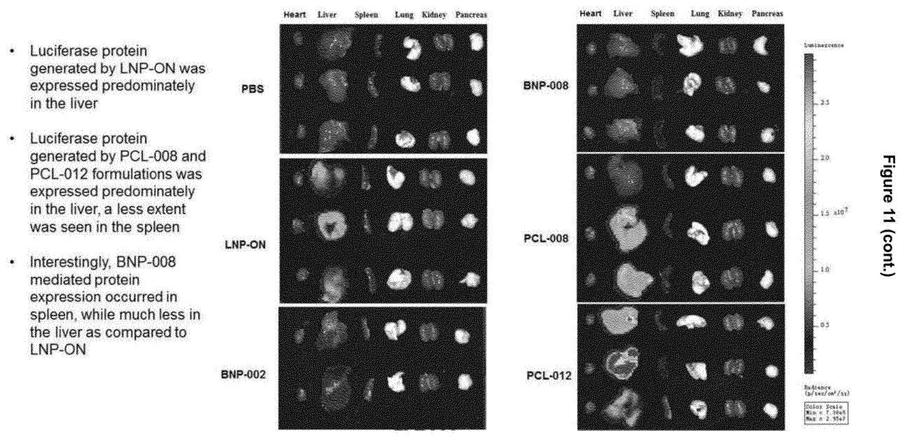

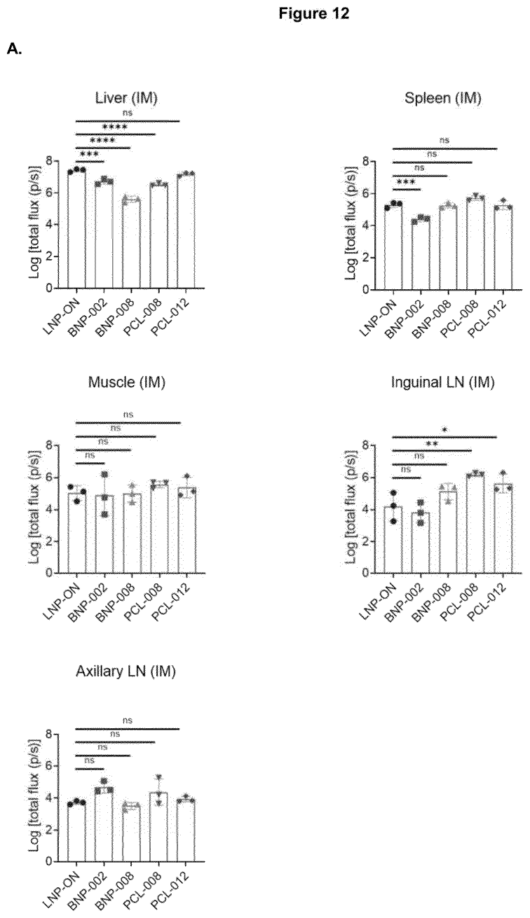

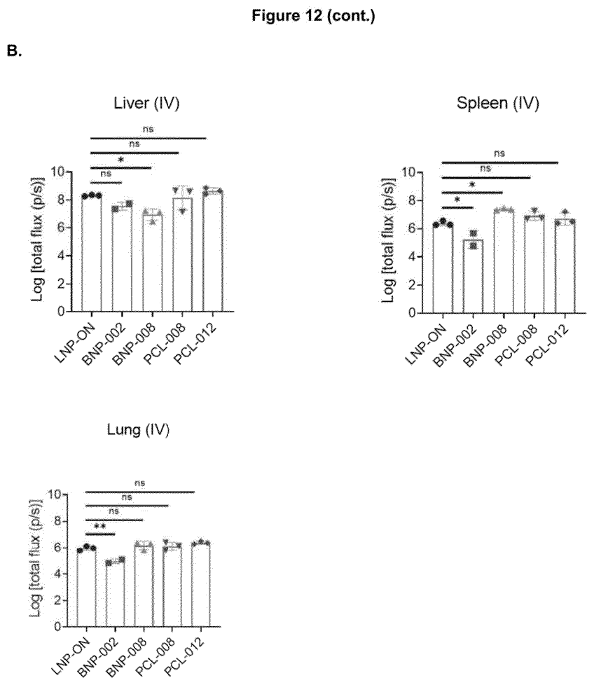

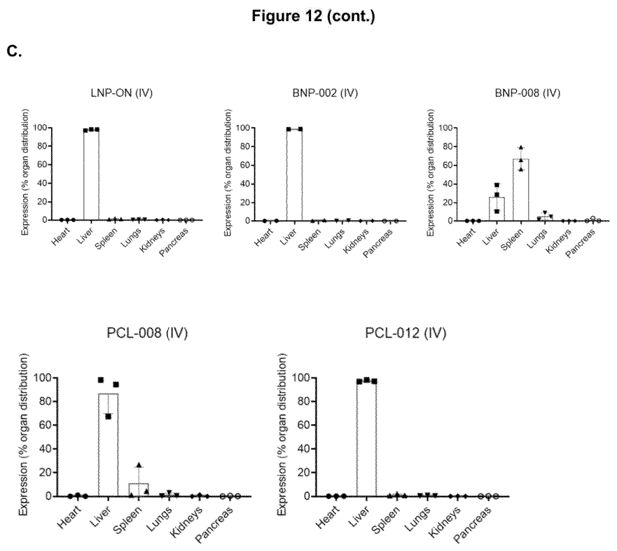

In vivo delivery efficacy of BNPs and PCLs was evaluated in C57BL/6 mice via different administration routes (IM, SC and IV). For IM administration routes, mice were randomly assigned into six different groups (3 mice in each group): control group (1×PBS), BNP-002, BNP-008, PCL-008 and PCL-012. All formulations were adminstrated via intramuscle (IM) route at the thigh muscle region at dosage of 0.35 mg/kg. Strong expression of luciferase protein was observed at the injection site and upper abdomen in the mice 6 h after IM injection (FIG. 11A). Ex vivo images of luminescence in major organs showed that abundant expression of Luc protein was seen at the liver in mice 6 h after injection, whereas less luminescent signal was detected in the popliteal and inguinal lymph nodes (FIG. 11B). Further ex vivo imaging quantitative analysis showed that there is no significant difference in terms of bioluminescent signal among different groups at injection site post 6 h administration (FIG. 12A. Moreover, significantly abundant expression of Luc protein was seen at the liver in mice 6 h after injection for LNP-ON as compared to ACM mRNA nanoparticles (FIG. 12A). Notably, PCL-008 and PCL-012 generated significantly higher expression of Luc protein at inguinal lymph nodes than LNP-ON after 6 h administration (p>0.05) (FIG. 12A. The results indicated that optimal BNPs and PCLs demonstrated potent in vivo mRNA delivery efficacy at inguinal lymph nodes via IM, yet with significant off target at liver as compared to LNP-ON.

Being encouraged by comparable Luciferase protein expression between Luc mRNA BNPs, PCLs and benchmark LNP-ONPs administrated via IM, we further investigated the effects of administration routes (subcutaneous (SC) and intravenous (IV)) of BNPs and PCLs for delivering of mRNA. SC administration of Luc mRNA resulted in protein expression mainly at the site of injection (data not shown). Strong bioluminescent signal was also observed at 6 hours post SC administration at upper abdomen for LNP-ONP LNP-ON (data not shown). Organs/tissues were then excised, and bioluminescence was further analyzed. Luciferase protein generated by LNP-ONP LNP-ON was expressed mainly in the liver (74%), a less extent was seen in injection site (16%) and lymph nodes. In contrast, Luciferase protein generated by ACM formulations of the present invention was expressed mainly in the injection site (55-83%). It is noteworthy that Luc protein expression level was similar in inguinal lymph nodes among LNP-ONP, BNP-008 and PCL-008 after 6 h SC administration.

When Luc mRNA nanoparticles were injected intravenously, strong bioluminescent signal was detected at liver for LNP-ONP, PCL-008 and PCL-012 (FIGS. 11C and 12C). Luciferase protein generated by LNP-ONP LNP-ON was expressed predominately in the liver (98%) (FIG. 12D). This result is consistent with Weissman's findings, where IV injection of Luc mRNA LNPs led to predominant protein production in the liver possibly due to passive ApoE-mediated liver targeting. Notably, BNP-008 mediated protein expression mainly occurred in the spleen (67%), while remarkably less in the liver (26%) as compared to LNP-ONP LNP-ON (98%) (FIG. 12D). Anderson et al. have reported an optimized C-35 formulation comprising C12-200, DOPE, Chol and C14-PEG2000 (35:16:46.5:2.5) generated luciferase protein predominately in the liver. The BNP-008 may be the key factor for tuning spleen specificity. Siegwart group has developed selective organ targeting (SORT) nanoparticles for tissue specific mRNA delivery, where charge-mediated targeting was achieved using LNPs, and mixing with a permanently cationic lipid (i.e. DOTAP) or anionic lipid (i.e. 18PA) to targeting liver, lung and spleen, respectively. However, BNP-008 had a net positive surface charge with zeta potential of 22.9 mV. On the other hand, like LNP-ONP, BNP-002 consisting of MC3 generated luciferase protein predominately in the liver (98%). Therefore, for BNP-008, PBD-PEO works collectively with C12-200, DOPE and Chol, achieving spleen targeting delivery of mRNA. To the best of our knowledge, this is the first finding on hybrid lipid nanoparticles achieving targeted mRNA delivery to the spleen.

### In Vivo Immunogenicity Study

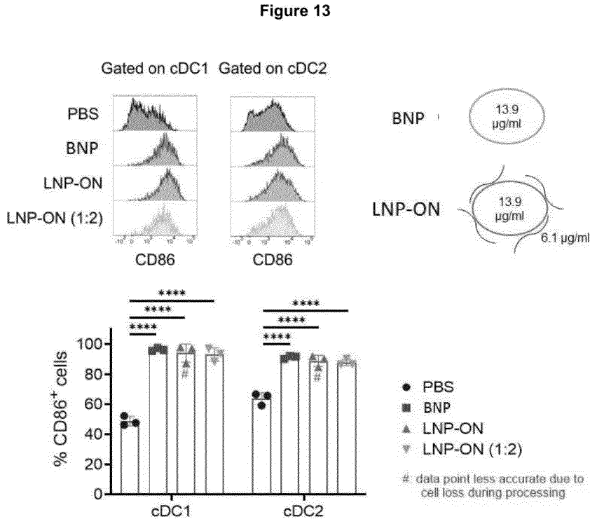

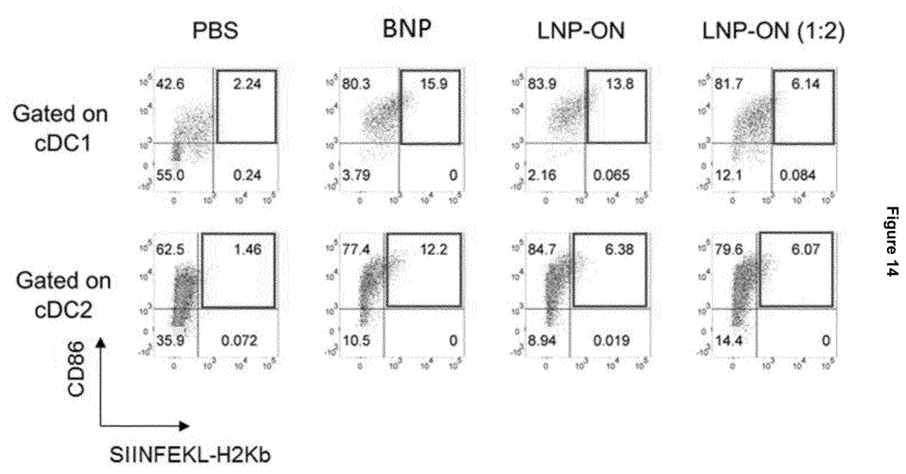

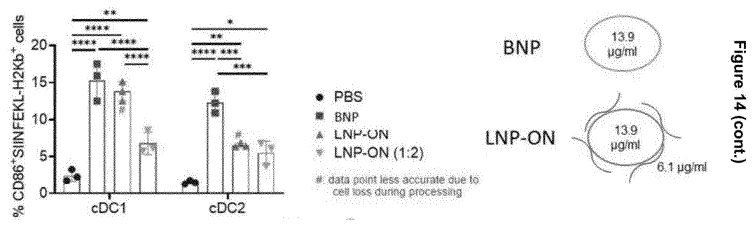

Classical dendritic cells consist of two subsets (cDC1 and cDC2) which are crucial for initiating the adaptive immune response. Two days after injection of LNP-mRNA or BNP-mRNA vaccine, strong upregulation of CD86 activation marker was seen on both cDC subsets (FIG. 13). Compared to PBS-injected controls, increase in CD86 was highly significant (FIG. 13), indicating both mRNA formulations were immuno-stimulatory. Next, we examined the DCs for surface expression of SIINFEKL peptide (SEQ ID NO: 4) presented in the context of MHC-II (H2kb), which would indicate successful translation of mRNA into OVA protein, followed by processing for MHC-II presentation. Both BNP-mRNA and LNP-mRNA formulations induced surface presentation of OVA peptides in cDC1 and cDC2 (FIG. 14). Moreover, the level of peptide expression induced by ACM-OVA mRNA was either similar or significantly higher than LNP-OVA mRNA (FIG. 14). Altogether, our results indicated that ACM-mRNA formulation strongly activated cDC1 and cDC2 in the lymph nodes, and the mRNA was translated and processed for surface presentation. Since lymph nodes are the principal sites of induction for the adaptive immune response, our findings strongly suggested that ACM-OVA mRNA would likely induce OVA-specific immunity.

ACM-OVA mRNA Vaccine Adaptive Immunity Study

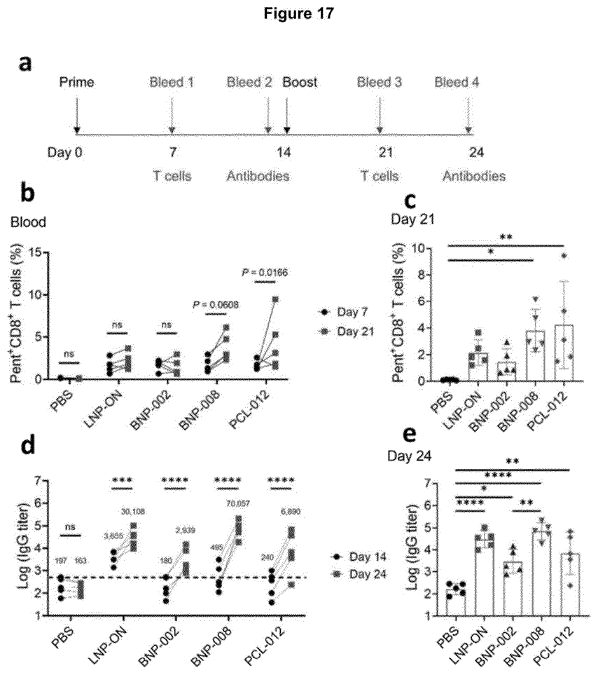

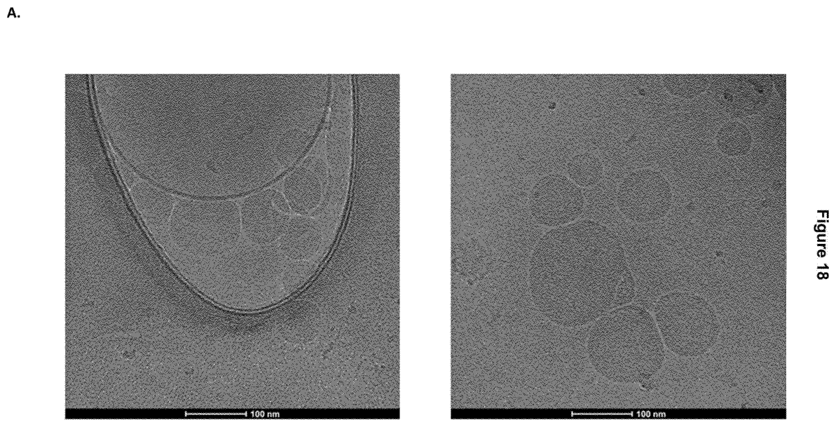

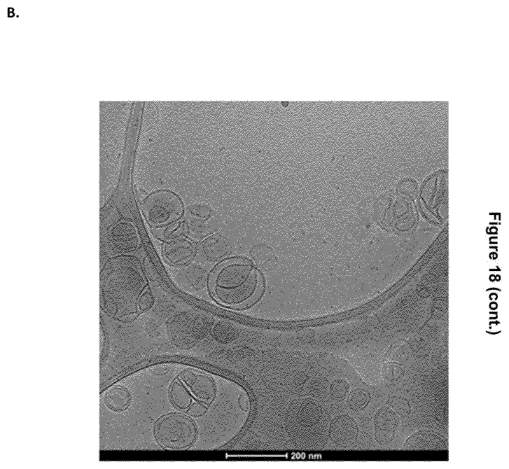

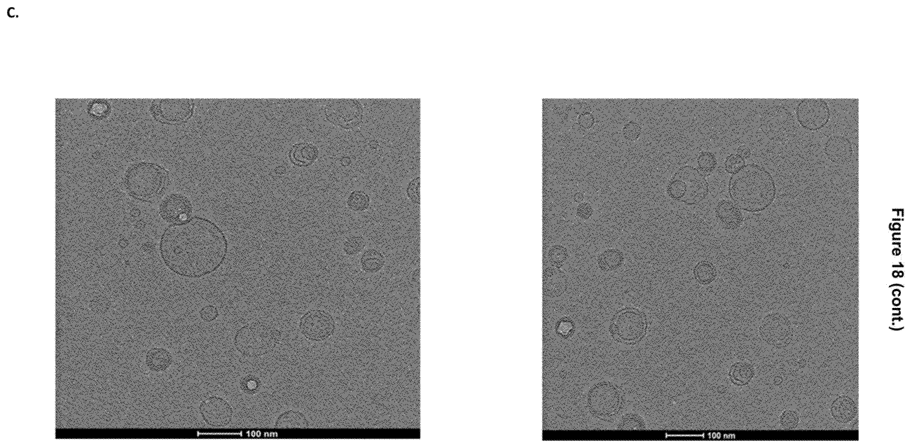

Mice were IM injected twice with LNP, BNP or PCL formulation encapsulating 5 μg per mouse of OVA mRNA (FIG. 17a). Circulating OVA-specific CD8+ T cells were identified using SIINFEKL pentamer. Immunisation with any formulation generated Pent+CD8+ T cells at ˜1.7% (FIG. 17b). Second dose of LNP-ON or BNP-002 failed to cause an increase in frequency, whereas BNP-008 or PCL-012 resulted in near significant and significant increase, respectively, in T cell frequency. On Day 21, BNP-008 and PCL-012 induced significantly higher Pent+CD8+ T cells than PBS controls (FIG. 17c). For OVA IgG titre, single dose of any formulation resulted to weak or undetectable titre (FIG. 17d). Second dose resulted in highly significant increase in IgG across all formulations. On Day 24, highest OVA IgG was observed with LNP or BNP-008 (FIG. 17e). Among the ACM formulations, BNP-008 consistently generated comparable CD8+ T cell and IgG response as LNP controls.

### CONCLUSION

In conclusion, a new class of polymer and lipid hybrid nanoparticles have been developed for mRNA delivery. The optimal formulation exhibited favourable physicochemical properties and superior encapsulation efficiency (˜100%). In comparison to benchmark LNP-ON, the optimal formulation outperformed with enhanced in vitro transfection efficacy and long term thermostability, as evidenced by high levels of Luc protein expression and OVA protein expression. Moreover, the ACM formulations displayed less cytotoxicity as compared to benchmark LNP-ON. Importantly, the optimal formulation demonstrated potent in vivo mRNA delivery efficacy, which is comparable to that of benchmark LNP-ON. Furthermore, OVA mRNA formulation strongly activated cDC1 and cDC2 in the lymph nodes to promote antigen surface presentation. BNP-008 consistently generated comparable CD8+ T cell and IgG response as LNP controls. The findings strongly suggested that ACM-OVA mRNA would likely induce OVA-specific adaptive immunity. Taken together, our work reports a novel class of polymer lipid hybrid nanoparticles with efficient protein and antigen expression as well as enhanced thermostability, which hold great potential for delivery of therapeutic mRNA over a wide range of diseases.

### Example 2: Further Characterisation of Polymer-Lipid Hybrid Nanoparticles Comprising a Lipid and a Block Copolymer and Uses Thereof for Organ Specific Delivery of mRNA to Liver, Spleen and Lung

1,2-di-O-octadecenyl-3-trimethylammonium propane (chloride salt) (DOTMA, Avanti) was bought from Merck, all the other chemicals and reagents as well as methods have been described above. Accordingly, if not specified otherwise materials and methods were as described above for example 1. In this example Luc mRNA loaded nanoparticles were prepared by microfluidizer according to Table 8, wherein for mRNA loaded polymer lipid hybrid nanoparticles mRNA was produced in a molar composition as presented in Table 8 by microfluidizer, where N/P molar ratio is 10:

TABLE 8
Exemplary Luc-mRNA loaded nanoparticles preparation by microfluidizer

| Formulation Name | BNP-002.2 | | BNP-012 | | BNP-025 | |
|---|---|---|---|---|---|---|
| mRNA source | Luciferase mRNA from ApexBio (1 mg/ml in 1 mM Sodium Citrate, pH 6.4) | | | | | |
| Polymer & Lipids | Dlin-MC3-DMA (Target | | PBD-b-PEO (Shilpa, | | PBD-b-PEO (Shilpa, Batch | |
| source | Mol), DSPC (Avanti | | Batch 45A), DOTAP | | 45B), DOTMA (Avanti | |
| | Lipids), PBD-b-PEO | | (Lipoid), Cholesterol | | Lipids), Cholesterol | |
| | (Shilpa, Batch 45A), | | (Corden Pharma), DSPC | | (Corden Pharma), DSPC | |
| | Cholesterol (Corden | | (Avanti Lipids) | | (Avanti Lipids) | |
| | Pharma) | | | | | |
| Polymer & Lipids | 5 mM (Molar %) of DLin-MC3- | | 10 mM (Molar %) of | | 10 mM (Molar %) of | |
| Composition | DMA:Cholesterol:DSPC:PBD- | | DOTAP:Cholesterol:DSPC:PBD- | | DOTMA:Cholesterol:DSPC:PBD- | |
| | b-PEO (49.3:39.0:10.1:1.6) | | b-PEO (40:48:10:2) | | b-PEO (40:48:10:2) | |
| Encapsulation | 20 mM Acetate buffer, pH 5.0 | | | | | |
| Buffer | | | | | | |
| Composition | | | | | | |
| Final Buffer | 20 mM Tris buffer, 4.5 mM Acetate, 5% Sucrose, pH 7.4 | | | | | |
| Composition | | | | | | |
| mRNA Starting | 244 | μg/mL | 130 | μg/mL | 130 | μg/mL |
| Concentration | | | | | | |
| N/P ratio | 10 | | 10 | | 10 | |
| Dialysis | 1000 Kda CE Tubing, 4 C, Overnight, sample:buffer (1:300) | | | | | |
| Batch size | 1 × 2.5 | mL | 1 × 3.5 | mL | 1 × 3.5 | mL |

After that, the polymer-lipid hybrid nanoparticles were characterized as shown in Table 9:

TABLE 9
Physiochemical characterization of Luc-mRNA loaded
nanoparticles prepared by microfluidizer.

| Test Sample Name | BNP-002.2 | | BNP-012 | | BNP-025 | |
|---|---|---|---|---|---|---|
| Appearance | Off-white suspension | | Off-white suspension | | Off-white suspension | |
| Particle Size (nm) | 92.99 | nm | 51.64 | nm | 52.34 | nm |
| Polydispersity Index | 0.097 | | 0.188 | | 0.198 | |
| Zeta Potential (mV) | 12.6 | mV | 45.6 | mV | 47.6 | mV |
| mRNA Loading | 266.5 μg/mL (97%) | | 164.4 μg/mL (98%) | | 163.3 μg/mL (97%) | |
| (μg/mL)/% EE | | | | | | |
| Transfection (Y/N) | Y | | Y | | Y | |
| Agarose Gel | Intact band | | Intact band | | Intact band | |
| Endotoxin (EU/mL) | 2.7 | EU/mL | 2.2 | EU/mL | 1.7 | EU/mL |

Accordingly, BNP-002.2 showed 93 nm in z-average diameter with lower polydispersity. BNP-012 had a particle size of 51 nm with a polydispersity of 0.188. BNP-025 exhibited a particle size of 52 nm and a polydispersity of 0.198. BNP-012 and BNP-025 displays zeta potential of ˜ 45 mV. Finally, all formulations demonstrated high Luc-mRNA encapsulation efficiency (>90%).

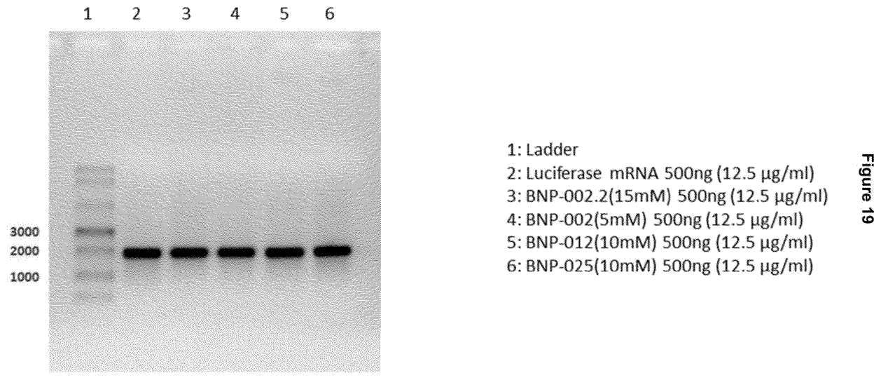

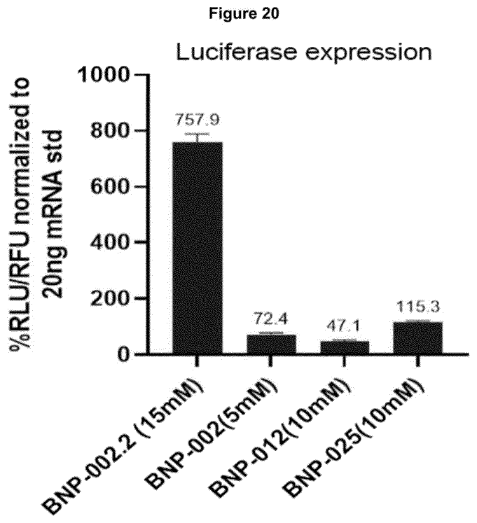

After that, exemplary Cryo-TEM images were produced for BNP-002.2, BNP-012 and BNP-025 (all loaded with Luciferase mRNA) (FIG. 18), in which BNP-002.2 showed spherical nanoparticles (50˜150 nm) with amorphous structure; BNP-012 showed ppredominant distribution: Multi-compartmental structure and vesicles consist of heterogeneous structure (i.e. vesicles fusion; vesicles with buddy, vesicles buddy surrounding by bilayer); BNP-025 showed vesicle structure (30-150 nm) with relatively higher polydispersity.

After that, Luc mRNA encapsulation efficiency was measured using Ribogreen Assay as shown in Table 10:

TABLE 10
Luc mRNA encapsulation efficiency measured
using Ribogreen Assay, wherein:

| | Conc. | Conc. | | | |
|---|---|---|---|---|---|
| | without Tx | with Tx | Loading | | % Free |
| Sample | (μg/ml) ¹ | (μg/ml) ² | (μg/ml) ³ | % EE⁴ | mRNA |
| BNP-002.2 (15 | 9.6 | 276.2 | 266.5 | 97 | 3 |
| mM), SF | | | | | |
| BNP-012(10 | 3.8 | 168.2 | 164.4 | 98 | 2 |
| mM), SF | | | | | |
| BNP-025(10 | 4.6 | 167.9 | 163.3 | 97 | 3 |
| mM), SF | | | | | |

¹ This is to measure any free un-encapsulated mRNA or mRNA that binds to the surface of the vesicles.
² This is to measure the total mRNA present in the sample as Triton was added to break open the vesicle.
³ mRNA loading is calculated by using the concentration with Triton minus the concentration without Triton.
⁴% EE is calculated by using Loading divided by concentration with Triton into percentage.

Accordingly, Ribogreen assay indicated that all formulations exhibited high encapsulation efficiency (>90%).

After that, an agarose gel image for Luc mRNA loaded nanoparticles prepared by microfluidizer were carried out indicating that all samples contain intact mRNA as no degradation observed in the gel (FIG. 19).

This was followed by an in vitro Luciferase mRNA nanoparticles transfection efficiency profiles in HEK293T cells indicating that all formulations showed high expression of luciferase protein, which was comparable to that of LNP-ON and that BNP-002.2 demonstrated remarkably high in vitro transfection potency as compared to LNP-ON (p<0.05).

After that, an endotoxin (Lonza LAL Assay) analysis of Luc mRNA nanoparticles was carried out as shown in Table 11, wherein the endotoxin levels of and Luc mRNA nanoparticles were investigated using Lonza Kinetic Chromogenic LAL Assay according to the manufacturer instructions. It was shown that Spike recovery of all nanoparticles met the acceptable range and that the endotoxin level of all nanoparticles were around ˜ 2 EU/mL, which also met the requirements (<10 EU/mL).

TABLE 11
Endotoxin (Lonza LAL Assay) analysis
of Luc mRNA nanoparticles

| | | Final | Spike | Acceptable Range |
|---|---|---|---|---|
| No. | Sample | EU/ml | Recovery (%) | (50%-200%) |
| 1 | BNP002.2(15 mM) | 2.7 | 81 | Pass |
| 2 | BNP002(5 mM) | 2.7 | 118 | Pass |
| 3 | BNP012(10 mM) | 2.2 | 130 | Pass |
| 4 | BNP025(10 mM) | 1.7 | 102 | Pass |

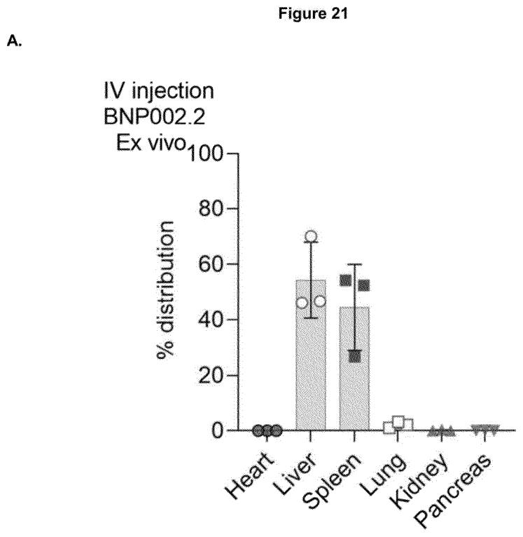

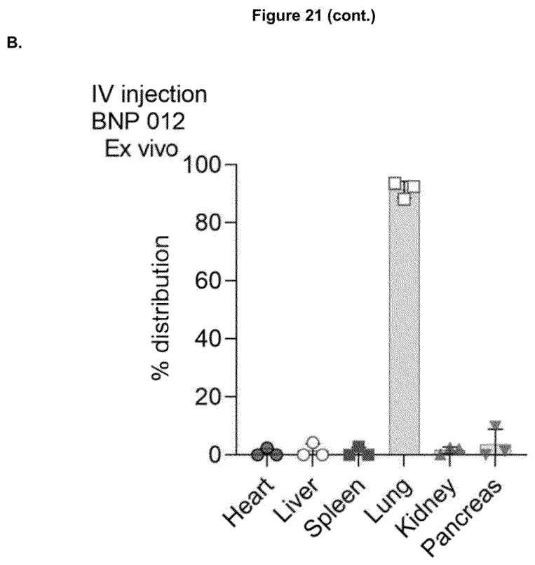

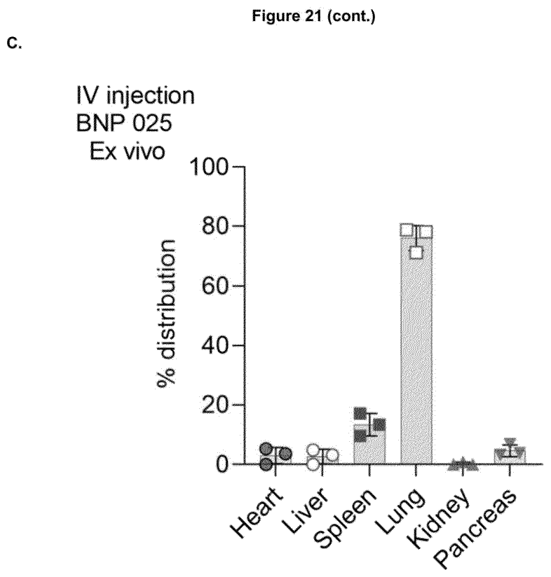

After that, Luciferase Protein expression Biodistribution Percentage Profile via IV Administration was carried out (FIG. 21). It was shown that BNP-002.2 yielded Luciferase protein accumulated in liver (54%), spleen (44.5%), 2.1% in the lung; BNP-012 led to Luciferase protein expression in liver (0.9%), while 1.4% in spleen, 92% in the lung; BNP-025 generated Luciferase protein in liver (2.7%), spleen (13%), 76% in the lung; BNP-012 and BNP-025 containing cationic lipids (DOTAP and DOTMA) generated luciferase protein predominately in the lung.

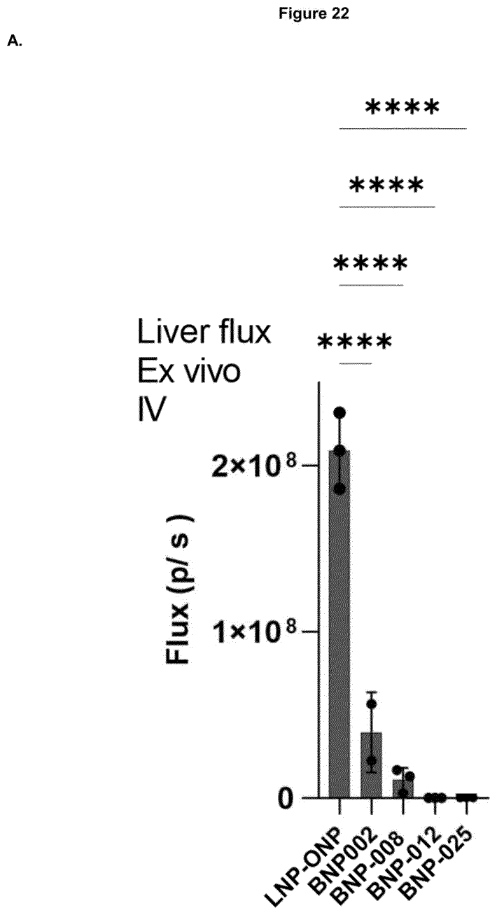

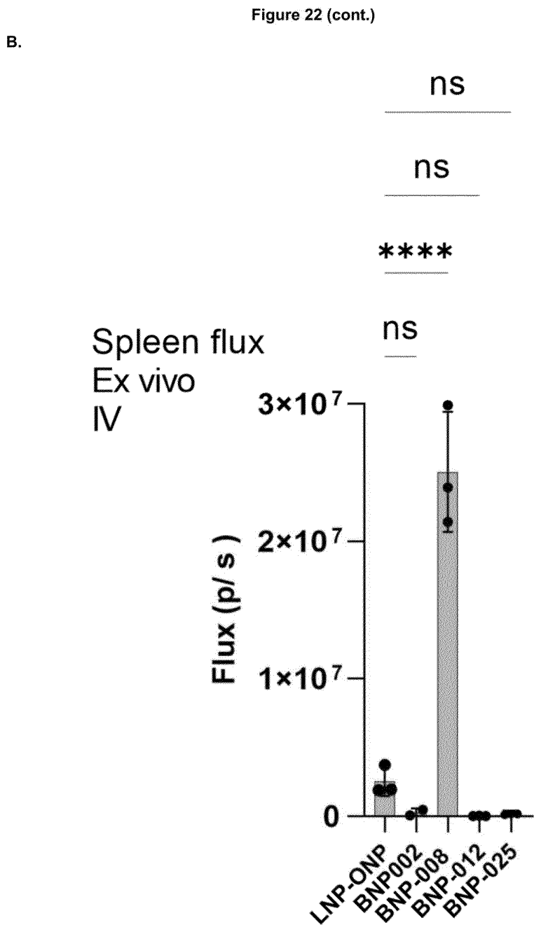

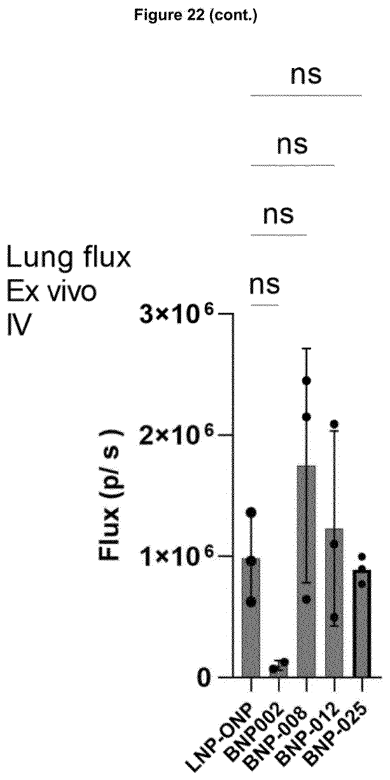

This was followed by Tissue Expression Profiles (Raw value of Flux) of the Luc mRNA-encoded Protein in Mice 6 h Post Administration via IV (FIG. 22) showing that LNP ONP produce significantly higher Luc protein than other groups at liver; BNP-008 yielded significant higher Luc protein than other groups at spleen; The amount of Luc protein expression at lung among difference groups was similar.

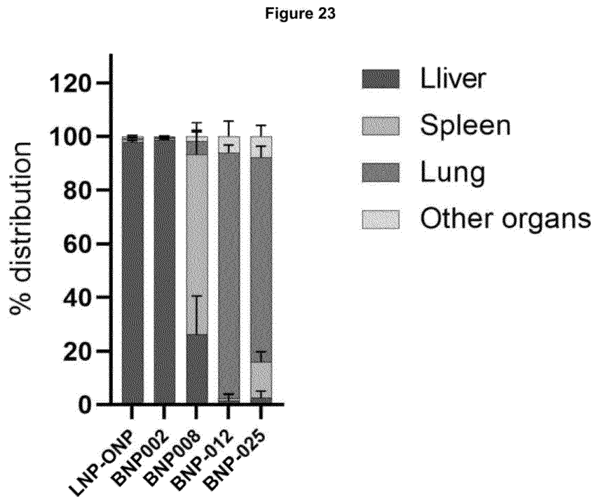

Finally, Luciferase Protein expression Biodistribution Percentage Profile (Flux) via IV Administration was performed (FIG. 23) showing that LNP-ON yielded Luciferase protein in liver (98%), while 1.2% in spleen, 0.5% in the lung; BNP-002 produced Luciferase protein in liver (98%), while 0.5% in spleen, 0.3% in the lung; BNP-008 facilitated higher levels of Luciferase protein expression in spleen (67%), liver (26%), 5% in the lung; BNP-012 generated Luciferase protein in spleen (1.4%), liver (0.9%), 92% in the lung; BNP-025 generated Luciferase protein in spleen (13%), liver (2.7%), 76% in the lung. Additionally, it was shown that PCL-008 as described elsewhere herein generated Luciferase protein mainly in liver (86.8%), while 10.7% in spleen, 1.3% in the lung and PCL-012 as described elsewhere herein produced Luciferase protein mainly in the liver (97.6%), less extent in spleen (1.3%) and lung (0.6%).

Conclusion: Organ Specific Delivery of mRNA to Liver, Spleen and Lung has been Achieved Via Engineered Block Copolymer Lipid Hybrid Nanoparticles of the Present Invention

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing “, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

1-38. (canceled)

39. A polymer-lipid hybrid nanoparticle comprising a lipid and a block copolymer, wherein the amount of said lipid, expressed in mole percentage present in the polymer-lipid hybrid nanoparticle, wherein the mole percentage refers to the total amount of all components that form the polymer-lipid nanoparticle, is greater than the amount of said block copolymer, expressed in mole percentage, present in the polymer-lipid hybrid nanoparticle, wherein said block copolymer is selected from a group consisting of:
a) Poly(butadiene)-poly(ethylene oxide) (PBD-PEO) block copolymer, preferably wherein said PBD-PEO diblock copolymer comprises 5-50 blocks PBD and 5-50 blocks PEO;
b) Poly(caprolactone)-poly(ethylene oxide) (PCL-PEO) block copolymer, preferably wherein said PCL-PEO diblock copolymer comprises 5-50 blocks PCL and 5-50 blocks;
c) Poly(lactide-co-glycolide)-poly(ethylene oxide) (PLGA-PEO) block copolymer, wherein said PLGA-PEO diblock copolymer comprises 5-50 blocks PLGA and 5-50 blocks PEO;
d) Polylactic acid-poly(ethylene oxide) (PLA-PEO) block copolymer, wherein said PLA-PEO diblock copolymer comprises 5-50 blocks PLA and 5-50 blocks PEO;
e) Poly(dimethylsiloxane)-poly(ethylene oxide) (PDMS-PEO) block copolymer or the triblock copolymer poly(2-methyloxazoline)-poly(dimethylsiloxane)-poly(2-methyloxazoline) (PMOXA-PDMS-PMOXA), where in PDMS copolymer comprises 5-60 blocks of PDMS, 5-50 blocks PEO, and 5-50 blocks of PMOXA;
f) PIP-PEO (polyisoprene-Polyethylene oxide);
g) Poly(butadiene)-poly(ethylene oxide) (PB-PEO) diblock copolymer;
h) Poly(dimethylsiloxane)-poly(ethylene oxide) (PDMS-PEO) diblock copolymer.

40. The polymer-lipid hybrid nanoparticle according to claim 39, wherein a mole % ratio of said lipid to said block copolymer is from about 31.8:12 to about 35:2.5, preferably said mole % ratio is selected from the group consisting of:
a) 49:12;
b) 35:2.5;
c) 31.8:12;
d) 35:12;
e) 23.8:4.8;
f) 45:10; and
g) 49:8.

41. The polymer-lipid hybrid nanoparticle according to claim 39, wherein said polymer-lipid hybrid nanoparticle having one or more of the following characteristics:
a) said polymer-lipid hybrid nanoparticle is synthetic;
b) a diameter greater than 75 nm, preferably said diameter ranging from about 80 nm to about 450 nm, further preferably said diameter ranging from about 80 nm to about 140 nm, most preferably said diameter ranging from about 100 nm to about 140 nm, wherein said diameter is determined by a dynamic light scattering (DLS) instrument using Z-average;
c) a polydispersity index (PDI) greater than about 0.15, preferably said PDI is greater than 0.17, further preferably said PDI is from about 0.175 to about 0.245;
d) a zeta potential between about-40 mV and about +40 mV, preferably said zeta potential is greater than 12 mV;
e) spherical particle exhibiting stacked bilayer structure;
f) having electro-lucent amorphous internal structure surrounded by a peripheral bilayer;
g) spherical particle with amorphous structure; and/or
h) vesicle with heterogeneous structure surrounded by a bilayer.

42. The polymer-lipid hybrid nanoparticle according to claim 39, wherein said polymer-lipid hybrid nanoparticle further comprising a stabilizer, preferably said stabilizer comprising cholesterol.

43. The polymer-lipid hybrid nanoparticle according to claim 39, wherein said polymer-lipid hybrid nanoparticle further comprising a stabilizer, wherein said stabilizer is selected from the group consisting of: cholesterol and hydroxycholesterol.

44. The polymer-lipid hybrid nanoparticle according to claim 39, further comprising another lipid, preferably said lipid is cationic, ionizable cationic lipid and/or anionic lipid selected from a group consisting of:
a) 1,2-Dimyristoyl-sn-glycero-3-phosphocholine (DMPC, 14:0 PC), preferably having Formula II: 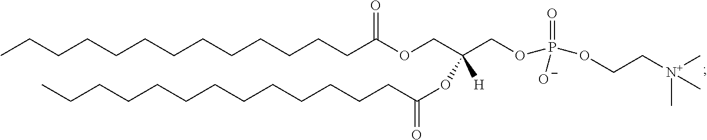 [SMILES: CCCCCCCCCCCCCC(=O)OC[C@H](COP(=O)([O-])OCC[N+](C)(C)C)OC(=O)CCCCCCCCCCCCC]
b) 1,2-Distearoyl-sn-glycero-3-phosphocholine (DSPC, 18:0 PC), preferably having Formula III: 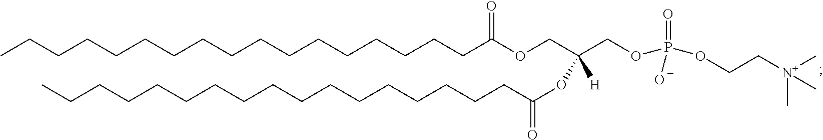 [SMILES: CCCCCCCCCCCCCCCCCC(=O)OC[C@H](COP(=O)([O-])OCC[N+](C)(C)C)OC(=O)CCCCCCCCCCCCCCCCC]
c) 1,2-dioleoyl-sn-glycero-3-phosphoethanolamine (DOPE), preferably having Formula IV: 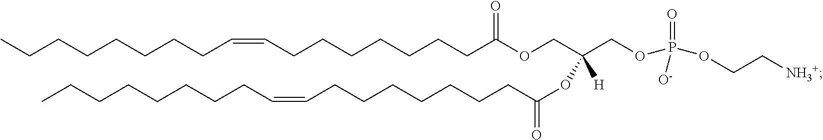
d) 1,2-dioleoyl-3-trimethylammonium-propane (DOTAP), preferably having Formula V: 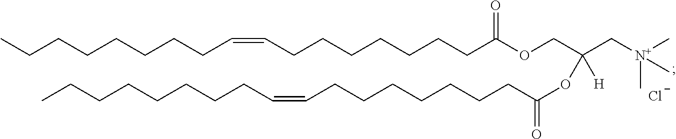 [SMILES: CCCCCCCC/C=C\CCCCCCCC(=O)OCC(C[N+](C)(C)C)OC(=O)CCCCCCC/C=C\CCCCCCCC ; [Cl-]]
e) 1,2-Dioleoyl-3-trimethylammonium propane (DODAP), preferably having Formula VI: 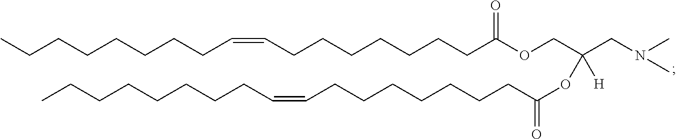 [SMILES: CCCCCCCC/C=C\CCCCCCCC(=O)OCC(CN(C)C)OC(=O)CCCCCCC/C=C\CCCCCCCC]
f) 1,2-di-O-octadecenyl-3-trimethylammonium propane (DOTMA), preferably having Formula VII: 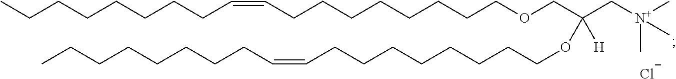 [SMILES: CCCCCCCC/C=C\CCCCCCCCOCC(C[N+](C)(C)C)OCCCCCCCC/C=C\CCCCCCCC ; [Cl-]]
g) 1,2-dioleyloxy-3-dimethylaminopropane (DODMA), e.g., having Formula VII: 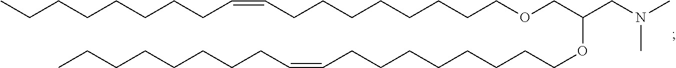 [SMILES: CCCCCCCC/C=C\CCCCCCCCOCC(CN(C)C)OCCCCCCCC/C=C\CCCCCCCC]
h) Dimethyldioctadecylammonium (DDA), preferably having Formula IX: 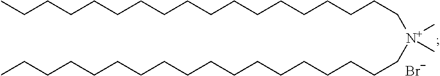 [SMILES: CCCCCCCCCCCCCCCCCC[N+](C)(C)CCCCCCCCCCCCCCCCCC ; [Br-]]
i) 1,2-dioleoyl-sn-glycero-3-phosphate (18:1 PA), preferably having Formula X: 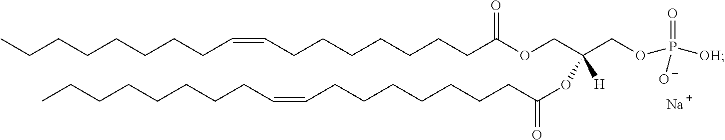 [SMILES: CCCCCCCC/C=C\CCCCCCCC(=O)OC[C@H](COP(=O)([O-])O)OC(=O)CCCCCCC/C=C\CCCCCCCC ; [Na+]]
j) 1,2-dimyristoyl-sn-glycero-3-phosphate (14:0 PA), preferably having Formula XI: 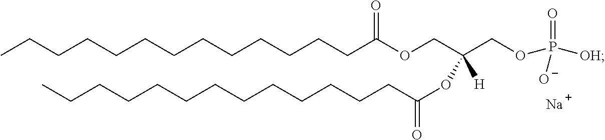 [SMILES: CCCCCCCCCCCCCC(=O)OC[C@H](COP(=O)([O-])O)OC(=O)CCCCCCCCCCCCC ; [Na+]]
k) bis(monooleoylglycero)phosphate (18:1 BMP), preferably having Formula XII:

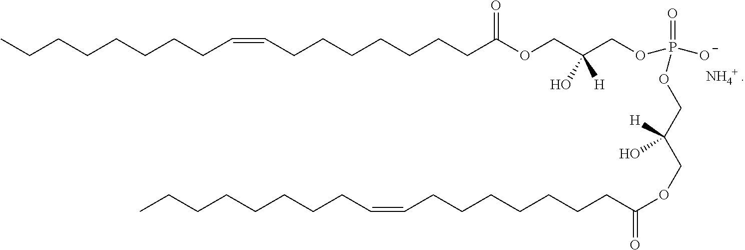

45. The polymer-lipid hybrid nanoparticle according to claim 39, wherein said lipid is selected from a group consisting of:
a) an ionizable lipid DLin-MC3-DMA (MC3 or (6Z,9Z,28Z,31Z)-Heptatriaconta-6,9,28,31-tetraen-19-yl 4-(dimethylamino) butanoate) having Formula XIII: 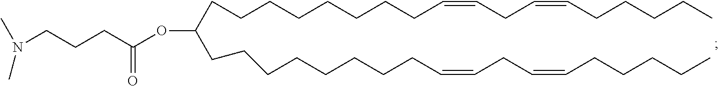 [SMILES: CCCCC/C=C\C/C=C\CCCCCCCCC(CCCCCCCC/C=C\C/C=C\CCCCC)OC(=O)CCCN(C)C]
b) an ionizable lipid C12-200, preferably having Formula XIV: 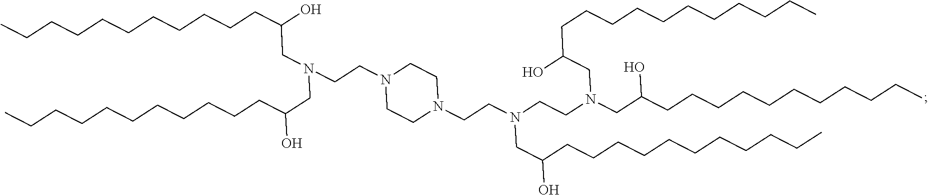 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]
c) an ionizable lipid 3060i10, preferably having Formula XVIII:

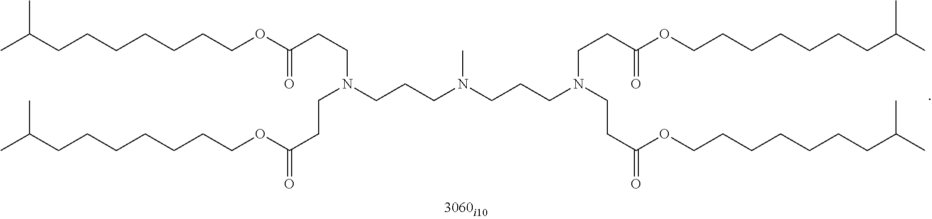 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C]

46. The polymer-lipid hybrid nanoparticle according to claim 39, wherein said block copolymer is selected from a group consisting of:
a) PBD-PEO block copolymer, wherein said PBD-PEO diblock copolymer comprises 5-50 blocks PBD and 5-50 blocks PEO, wherein said PBD-PEO block copolymer is PBD_{1.2k}-b-PEO_{0.6k}, wherein k-1000 Da, preferably having Formula XV: 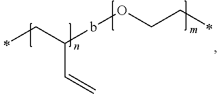
wherein n=22; b denotes block, m=12; *=OCH3;
b) PCL-PEO block copolymer, wherein said PCL-PEO diblock copolymer comprises 5-50 blocks PCL and 5-50 blocks PEO, wherein said PCL-PEO block copolymer is PCL_{3.3k}-b-PEO₁ₖ, k=1000 Da, preferably having Formula XVI: 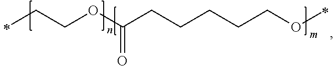
wherein n=22; m=29; *=H;
c) PLGA-PEO block copolymer, wherein said PLGA-PEO di-block copolymer comprises 5-50 blocks PLGA and 5-50 blocks PEO, wherein said PLGA-PEO block copolymer is PLGA_{1.9k}-b-PEO₁ₖ, k=1000 Da, preferably having Formula XVI: 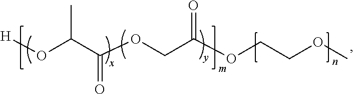
wherein x=23; y=4; m=26; n=29;
d) PLA-PEO block copolymer, wherein said PLA-PEO di-block copolymer comprises 5-50 blocks PLA and 5-50 blocks PEO;
e) PDMS-PEO block copolymer or the triblock copolymer PMOXA-PDMS-PMOXA, where in PDMS copolymer comprises 5-60 blocks of PDMS, 5-50 blocks PEO, and 5-50 blocks of PMOXA;
f) PIP-PEO (polyisoprene-Polyethylene oxide);
g) Poly(butadiene)-poly(ethylene oxide) (PB-PEO) diblock copolymer; and
h) Poly(dimethylsiloxane)-poly(ethylene oxide) (PDMS-PEO) diblock copolymer.

47. The polymer-lipid hybrid nanoparticle according to claim 39, wherein said polymer-lipid hybrid nanoparticle comprising or consisting of:
a) PBD-PEO, MC3 and CHOL, preferably at about a following mole ratio: 12:49:39;
b) PBD-PEO, C12-200 and CHOL, preferably at about a following mole ratio: 12:49:39;
c) PBD-PEO, DOPE, C12-200 and CHOL, preferably at about a following mole ratio: 2.5:16:35:46.5;
d) PBD-PEO, DOPE, C12-200 and CHOL, preferably at about a following mole ratio: 12:12.8:31.8:43.4;
e) PBD-PEO, DOPE, C12-200 and CHOL, preferably at about a following mole ratio: 12:6.5:35:46.5;
f) PBD-PEO, DOPE, C12-200 and CHOL, preferably at about a following mole ratio: 4.8:23.8:23.8:47.6;
g) DMG-PEG, DSPC, MC3 and CHOL, preferably at about a following mole ratio: 1.6:10.1:49.3:39.0;
h) PCL-PEO, DMPC, MC3 and CHOL, preferably at about a following mole ratio: 10:10:45.0:35.0;
i) PCL-PEO, DMPC, MC3 and CHOL, preferably at about a following mole ratio: 10:10:45.0:35.0;
j) PCL-PEO, DMPC, MC3 and CHOL, preferably at about a following mole ratio: 8:4:49.0:39.0;
k) PCL-PEO, DMPC, MC3 and CHOL, preferably at about a following mole ratio: 8:4:49.0:39.0;
l) DLin-MC3-DMA: Cholesterol: PBD-b-PEO, preferably at about a following mole ratio: 49:39:12;
m) DLin-MC3-DMA: Cholesterol: DSPC: PBD-b-PEO, preferably at about a following mole ratio: 49.3:39.0:10.1:1.6;
n) DOTAP: Cholesterol: DSPC: PBD-b-PEO, preferably at about a following mole ratio: 40:48:10:2;
o) DOTMA: Cholesterol: DSPC: PBD-b-PEO, preferably at about a following mole ratio: 40:48:10:2.

48. The polymer-lipid hybrid nanoparticle according to claim 39, further comprising a soluble encapsulated antigen, wherein said soluble encapsulated antigen is a protein and/or polynucleotide, preferably said protein is a nuclease involved in a gene- or RNA-editing, the polynucleotide is selected from a RNA, guide RNA, self-amplifying mRNA (saRNA), an antisense oligonucleotide molecule or a DNA molecule.

49. The polymer-lipid hybrid nanoparticle according to claim 39, wherein said nanoparticle is capable of selectively targeting tissues and/or organs, wherein said nanoparticle is not functionalized or attached to any ligand capable of targeting and/or binding to said target tissues and organs.

50. The polymer-lipid hybrid nanoparticle according to claim 39, wherein said polymer-lipid hybrid nanoparticle is not a polymersome.

51. The polymer-lipid hybrid nanoparticle according to claim 39, wherein said polymer-lipid hybrid nanoparticle is selected from the group consisting of:
a) BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2);
b) BNP-002 having 5 mM (Molar %) of DLin-MC3-DMA: Cholesterol: PBD-b-PEO (49:39:12);
c) BNP-002.2 having 5 mM (Molar %) of DLin-MC3-DMA: Cholesterol: DSPC: PBD-b-PEO (49.3:39.0:10.1:1.6);
d) PCL-008 having PCL-PEO: DMPC: MC3: CHOL (10:10:45.0:35.0); and
e) PCL-012 having PCL-PEO: DMPC: MC3: CHOL (8:4:49.0:39.0).

52. The polymer-lipid hybrid nanoparticle according to claim 39, wherein said polymer-lipid hybrid nanoparticle is capable of targeting a tissue/s and/or cell/s of an organ selected from the group consisting of: liver, spleen and lung/s, preferably said targeting is carried out without using a functional ligand/s; further preferably:
a) for said lung/s targeting the polymer-lipid hybrid nanoparticle/s is BNP-012 having 10 mM (Molar %) of DOTAP: Cholesterol: DSPC: PBD-b-PEO (40:48:10:2) and/or BNP-025 having 10 mM (Molar %) of DOTMA: Cholesterol: DSPC: PBD-b-PEO (40:48:10:2);
b) for said liver targeting the polymer-lipid hybrid nanoparticles is BNP-002 having 5 mM (Molar %) of DLin-MC3-DMA: Cholesterol: PBD-b-PEO (49:39:12), PCL-008 having PCL-PEO: DMPC: MC3: CHOL (10:10: 45.0:35.0) and/or PCL-012 having PCL-PEO: DMPC: MC3: CHOL (8:4:49.0:39.0);
c) for said spleen targeting the polymer-lipid hybrid nanoparticles is BNP-002.2 having 5 mM (Molar %) of DLin-MC3-DMA: Cholesterol: DSPC: PBD-b-PEO (49.3:39.0:10.1:1.6).

53. A composition or kit comprising a polymer-lipid hybrid nanoparticle according to claim 39.

54. The composition according to claim 53, wherein said composition is a pharmaceutical or diagnostic composition.

55. The composition according to claim 53, wherein said composition is a vaccine.

56. A method of eliciting an immune response in a subject, comprising:
i) providing a polymer-lipid hybrid nanoparticle of claim 39 or a composition comprising said polymer-lipid hybrid nanoparticle to said subject,
ii) administering said polymer-lipid hybrid nanoparticle or composition to said subject, preferably said administering is intradermal, intraperitoneal, intramuscular, subcutaneous, intravenous injection, or non-invasive administration to a mucosal surface.

57. A method of delivering nucleotide/s to inside a cell without using viral vector/s as delivery means, said method comprising:
i) providing a polymer-lipid hybrid nanoparticle of claim 39 or a composition comprising said polymer-lipid hybrid nanoparticle;
ii) contacting said polymer-lipid hybrid nanoparticle or composition with said cell.

58. A method of modifying and/or engineering and/or interfering with a genetic material or template/s inside a cell, comprising:
i) providing the polymer-lipid hybrid nanoparticle of claim 39 or a composition comprising said polymer-lipid hybrid nanoparticle;
ii) contacting said polymer-lipid hybrid nanoparticle or composition with said cell.
